(19) United States
(12) Patent Application Publication
(10) Pub. No.: US 2025/0177899 A1
(43) Pub. Date: Jun. 5, 2025

(54) AIR PURIFIER AND AIR PURIFYING APPARATUS COMPRISING SAME

(71) Applicant: Samsung Electronics Co., Ltd., Suwon-si (KR)

(72) Inventors: Seoyoung CHO, Suwon-si (KR); Juyoung KIM, Suwon-si (KR); Nakhyun KIM, Suwon-si (KR); Huiyoung KIM, Suwon-si (KR); Juwan PARK, Suwon-si (KR); Changhyun PARK, Suwon-si (KR); Changwoo JUNG, Suwon-si (KR); Sunghyun CHUN, Suwon-si (KR)

(21) Appl. No.: 19/042,671

(22) Filed: Jan. 31, 2025

Related U.S. Application Data
(63) Continuation of application No. PCT/KR2023/013226, filed on Sep. 5, 2023.

(30) Foreign Application Priority Data
Oct. 7, 2022 (KR) 10-2022-0129050
Dec. 9, 2022 (KR) 10-2022-0171862

(51) Int. Cl. B01D 46/00; A61L 9/20

(52) U.S. Cl. CPC B01D 46/0002; A61L 9/20; B01D 46/0032; B01D 46/0047; A61L 2209/12; A61L 2209/14; B01D 2265/021; B01D 2273/30; B01D 2279/50; B01D 2279/65

(57) ABSTRACT

An air purifier is provided. The air purifier includes a fan that generates airflow, a grill assembly arranged above the fan and forms an upper exterior of the air purifier, and a body frame configured to support the grill assembly, wherein the grill assembly includes a first grill frame having first grills arranged at first intervals (G1), a second grill frame having second grills arranged at second intervals (G2) that are smaller than the first intervals, and an edge frame overlapping an outer edge of the second grill frame in a vertical direction and being assembled onto an outer surface of the first grill frame, and wherein the grill assembly has a first rotation assembly structure configured to rotationally assemble the edge frame onto the first grill frame such that the edge frame is fixed in the vertical direction with respect to the first grill frame and rotational movement is restricted with respect to the first grill frame.

### CROSS-REFERENCE TO RELATED APPLICATION(S)

This application is a continuation application, claiming priority under § 365 (c), of an International application No. PCT/KR2023/013226, filed on Sep. 5, 2023, which is based on and claims the benefit of a Korean patent application number 10-2022-0129050, filed on Oct. 7, 2022, in the Korean Intellectual Property Office, and of a Korean patent application number 10-2022-0171862, filed on Dec. 9, 2022, in the Korean Intellectual Property Office, the disclosure of each of which is incorporated by reference herein in its entirety.

### BACKGROUND

### 1. Field

The disclosure relates to an air purifier and an air purifying apparatus including the same.

### 2. Description of Related Art

Air purifying apparatuses are designed to improve indoor air quality in various indoor spaces, such as homes or offices, and examples of air purifying apparatuses may include air purifiers for removing foreign substances, such as fine dust, through a filter.

An air purifier may include an introduction port through which polluted indoor air is introduced, a filter for purifying the polluted indoor air, and a discharge port through which the purified air is discharged. The air purifier may be equipped with a fan to generate airflow.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an air purifier and air purifying comprising same.

Additional aspects will be set forth in part in the description which follows and, in part, will be apparent from the description, or may be learned by practice of the presented embodiments.

In accordance with an aspect of the disclosure, an air purifier is provided. The air purifier includes a fan that generates air flow, a grill assembly arranged above the fan and forms an upper exterior of the air purifier, and a body frame configured to support the grill assembly, wherein the grill assembly includes a first grill frame having first grills arranged at first intervals (G1), a second grill frame having second grills arranged at second intervals (G2) that are smaller than the first intervals, and an edge frame overlapping an outer edge of the second grill frame in a vertical direction and being assembled onto an outer surface of the first grill frame, and wherein the grill assembly has a first rotation assembly structure configured to rotationally assemble the edge frame onto the first grill frame such that the edge frame is fixed in the vertical direction with respect to the first grill frame and rotational movement is restricted with respect to the first grill frame.

In accordance with another aspect of the disclosure, n air purifying apparatus including an air purifier configured to remove dust from air, and an air sterilizer configured to sterilize the air from which the dust has been removed is provided. The air purifier includes a fan generating airflow, a grill assembly arranged above the fan and forming an upper exterior of the air purifier, and a body frame configured to support the grill assembly, wherein the grill assembly includes a first grill frame having first grills arranged at first intervals, a second grill frame having second grills arranged at second intervals that are smaller than the first intervals, and an edge frame overlapping an outer edge of the second grill frame in a vertical direction and being assembled onto an outer edge of the first grill frame, and wherein the grill assembly has a first rotation assembly structure configured to rotationally assemble the edge frame onto the first grill frame such that the edge frame is fixed in the vertical direction with respect to the first grill frame and rotational movement is restricted with respect to the first grill frame.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating an air purifier according to an embodiment of the disclosure;

FIG. 2 is an exploded perspective view illustrating an air purifier according to an embodiment of the disclosure;

FIG. 3 is an exploded perspective view of a portion of an air purifier according to an embodiment of the disclosure;

FIG. 4 is a cross-sectional view of an air purifier according to an embodiment of the disclosure;

FIG. 5 is an exploded perspective view illustrating a configuration of a grill assembly according to an embodiment of the disclosure;

FIG. 6 is an assembled perspective view illustrating a configuration of a grill assembly according to an embodiment of the disclosure;

FIG. 7 is a perspective view illustrating a state before assembly of a first grill frame and an edge frame of a grill assembly according to an embodiment of the disclosure;

FIG. 8 is a perspective view illustrating a state after assembly of a first grill frame and an edge frame of a grill assembly according to an embodiment of the disclosure;

FIG. 9 is a cross-sectional view conceptually illustrating a state before assembly of a first grill frame and an edge frame of FIG. 7 according to an embodiment of the disclosure;

FIG. 10 is a cross-sectional view conceptually illustrating a state after assembly of a first grill frame and an edge frame of FIG. 8 according to an embodiment of the disclosure;

FIG. 11 is a diagram illustrating an operation for separating an edge frame from a first grill frame in a grill assembly according to an embodiment of the disclosure;

FIG. 12 is an exploded perspective view illustrating a structure for assembling a fan onto a grill assembly according to an embodiment of the disclosure;

FIG. 13 is an exploded perspective view illustrating a structure of assembling a duct frame onto a grill assembly of FIG. 12 according to an embodiment of the disclosure;

FIG. 14 is an assembled perspective view illustrating a structure of assembling a duct frame onto a grill assembly onto which a fan of FIG. 12 is assembled according to an embodiment of the disclosure;

FIG. 15 is a perspective view illustrating a duct frame according to an embodiment of FIG. 13 according to an embodiment of the disclosure;

FIG. 16 is a perspective view illustrating a first grill frame of a grill assembly according to an embodiment of FIG. 13 according to an embodiment of the disclosure;

FIG. 17 is a diagram illustrating an assembly structure of a grill assembly and a duct frame according to an embodiment of the disclosure;

FIG. 18 is an exploded perspective view illustrating a state before a grill assembly is assembled onto a body frame according to an embodiment of the disclosure;

FIG. 19 is an enlarged perspective view illustrating a portion of a grill assembly in FIG. 18 according to an embodiment of the disclosure;

FIG. 20 is an enlarged perspective view illustrating a portion of a body frame in FIG. 18 according to an embodiment of the disclosure;

FIG. 21 illustrates a state in which a grill assembly of FIG. 18 is seated on an inner frame according to an embodiment of the disclosure;

FIG. 22 illustrates a state in which a grill assembly of FIG. 21 is assembled onto an inner frame according to an embodiment of the disclosure;

FIG. 23 is a drawing illustrating a state in which a grill assembly is assembled onto a body frame in an air purifier according to an embodiment of the disclosure;

FIG. 24 is an exploded perspective view illustrating a state in which a cover frame is separated from an inner frame in an air purifier according to an embodiment of the disclosure;

FIG. 25 is a drawing illustrating a state in which a cover frame is assembled onto an inner frame in an air purifier of FIG. 23 according to an embodiment of the disclosure;

FIG. 26 is a perspective view illustrating an air purifying apparatus according to an embodiment of the disclosure;

FIG. 27 is an exploded perspective view illustrating an air sterilizer according to an embodiment of FIG. 26 according to an embodiment of the disclosure;

FIG. 28 is an exploded perspective view of a portion of an air sterilizer of FIG. 27 according to an embodiment of the disclosure;

FIG. 29 is an exploded perspective view of a connection part of an air purifying apparatus of FIG. 26 according to an embodiment of the disclosure;

FIG. 30 is a cross-sectional view of an air purifying apparatus of FIG. 26 according to an embodiment of the disclosure;

FIG. 31 is a perspective view illustrating an air purifier having a fixed guard, according to an embodiment of the disclosure;

FIG. 32 is a plan view of an air purifier according to an embodiment of FIG. 31 as viewed from a top of an air purifier according to an embodiment of the disclosure;

FIG. 33 is a cross-sectional view of a portion of an air purifier of FIG. 31 according to an embodiment of the disclosure;

FIG. 34 is an exploded perspective view of a fixed guard according to an embodiment of the disclosure;

FIG. 35 is an assembled perspective view of a fixed guard according to an embodiment of the disclosure;

FIGS. 36 and 37 illustrate cross-sectional views of a fixed guard according to various embodiments of the disclosure;

FIG. 38 is an exploded perspective view illustrating a state in which a fixed guard is separated from an intermediate duct according to an embodiment of the disclosure; and

FIG. 39 is a cross-sectional perspective view illustrating a state in which a fixed guard is assembled onto an intermediate duct according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Terms, such as “first” or “second” may be used to describe various elements, but the elements are not limited by the terms. These terms are only used to distinguish one element from another element. For example, a first element may be referred to as a second element, and a second element may be referred to as a first element in a similar manner, without departing from the scope of the disclosure. The term “and/or” includes any and all combinations of one or more of the associated listed items.

Terms used herein are for describing embodiments and are not intended to limit the scope of the disclosure. The singular expression also includes the plural meaning as long as it does not inconsistent with the context. As used herein, terms, such as “comprises,” “includes,” or “has” specify the presence of stated features, numbers, stages, operations, components, parts, or a combination thereof, but do not preclude the presence or addition of one or more other features, numbers, stages, operations, components, parts, or a combination thereof. Like reference numerals in the drawings indicate members that perform substantially the same functions.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

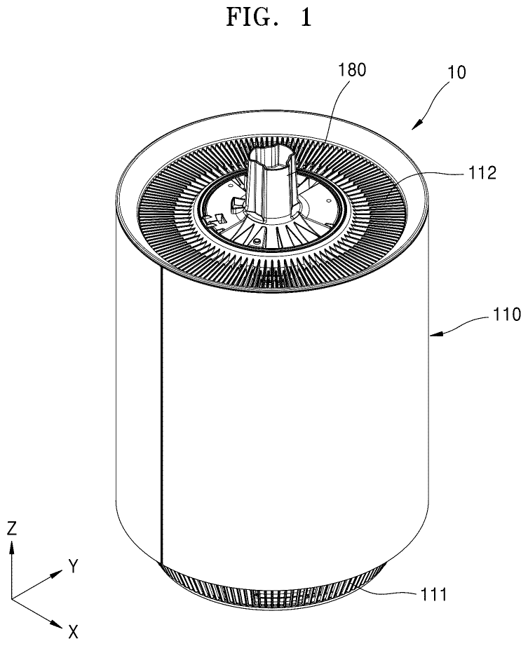

FIG. 1 is a perspective view illustrating an air purifier according to an embodiment of the disclosure.

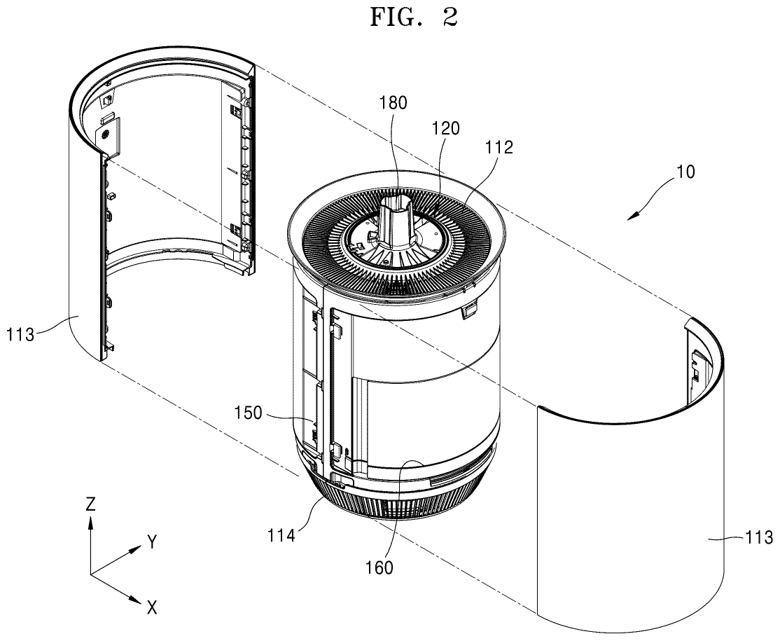

FIG. 2 is an exploded perspective view illustrating an air purifier according to an embodiment of the disclosure.

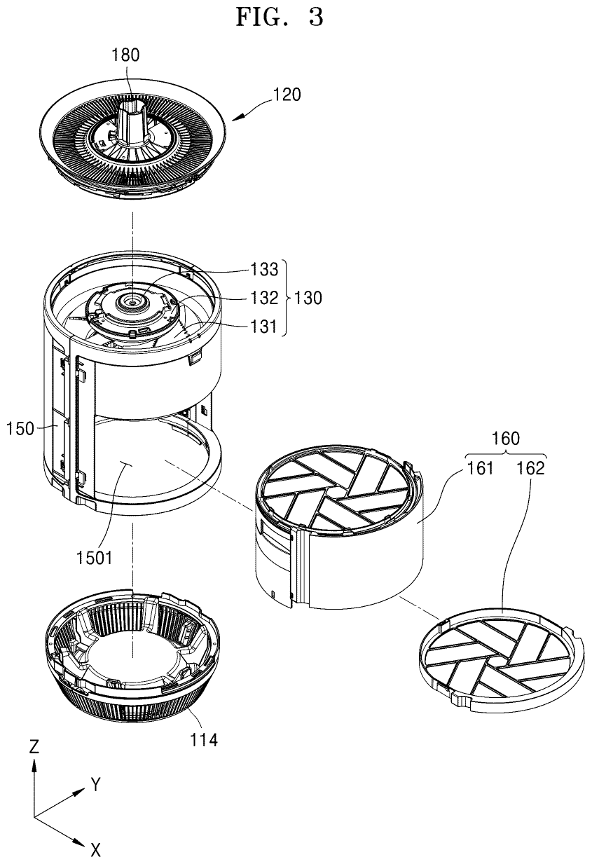

FIG. 3 is an exploded perspective view of a portion of an air purifier according to an embodiment of the disclosure.

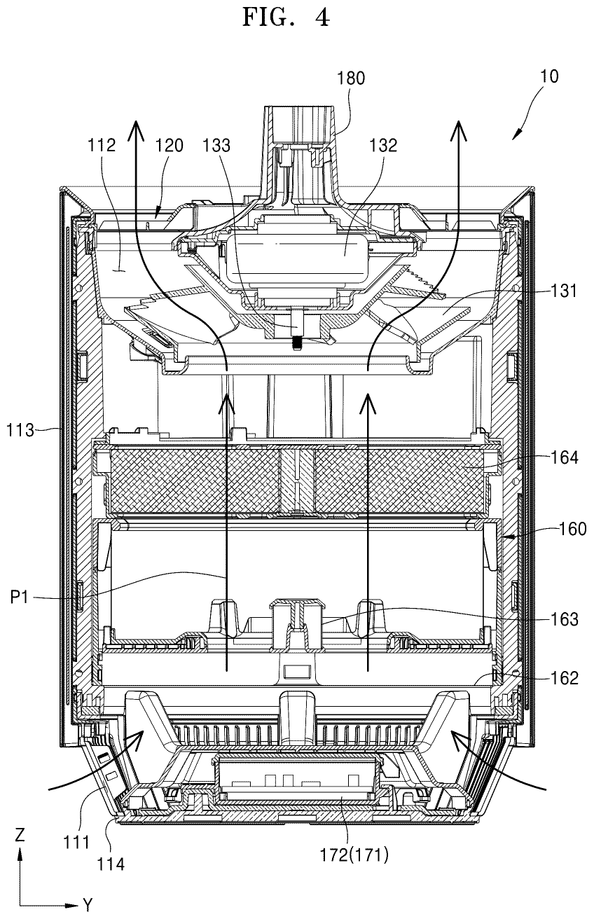

FIG. 4 is a cross-sectional view of an air purifier according to an embodiment of the disclosure.

Referring to FIGS. 1 to 4, the air purifier 10 according to an embodiment may include a housing 110 forming the exterior of the air purifier 10, and a body frame 150 provided to support various parts inside the housing 110.

The housing 110 has a first flow path P1 provided therein and may be configured to accommodate various parts of the air purifier 10.

The housing 110 may include an introduction port 111 and a discharge port 112. The introduction port 111 may be provided to allow air external to the housing 110 to flow into the housing 110. The discharge port 112 may be provided to allow air inside the housing 110 to be discharged to outside of the housing 110. The first flow path P1 extending from the introduction port 111 to the discharge port 112 may be provided inside the housing 110.

For example, the introduction port 111 may be formed in a lower portion of the housing 110. The discharge port 112 may be formed in an upper portion of the housing 110. At least a portion of the first flow path P1 may extend in a vertical direction, for example, in a Z direction. Accordingly, air introduced into the housing 110 through the introduction port 111 may move upward along the first flow path P1. However, the arrangement of the introduction port 111 and the discharge port 112 is not limited thereto, and the introduction port 111 and the discharge port 112 may be formed at various positions.

The housing 110 may include a housing body 113, a base 114, and a grill assembly 120. Each of the housing body 113, the base 114, and the grill assembly 120 may constitute a portion of the exterior of the air purifier 10.

The housing body 113 may form the side surface of the air purifier 10. The housing body 113 may be provided to cover various parts of the air purifier 10 in a horizontal direction. The housing body 113 may connect the base 114 to the grill assembly 120.

The housing body 113 may extend in a direction parallel to the first flow path P1. For example, the housing body 113 may extend in the vertical direction.

The housing body 113 may have a substantially cylindrical shape with a space therein. However, the shape of the housing body 113 is not limited thereto and the housing body 113 may have various shapes depending on the exterior of the air purifier 10.

The housing body 113 may be formed to be divided into a plurality of panels. The plurality of panels may be disassembled and assembled. The housing body 113 may be easily disassembled or assembled, and user accessibility to the components arranged inside the housing body 113 may be improved. However, the structure of the housing body 113 is not limited thereto and may be formed as an integrally formed single configuration.

The base 114 may be provided below the housing body 113. The base 114 may form the bottom surface of the air purifier 10. When the air purifier 10 is placed on the ground, the base 114 may support the air purifier 10. The base 114 may be coupled to a lower portion of the body frame 150.

A side surface of the base 114 may be formed in the circumferential direction of the base 114. The side surface of the base 114 may extend in the vertical direction or in a direction having a certain angle with the vertical direction.

The introduction port 111 may be formed in the base 114. The introduction port 111 may be formed on the side surface of the base 114. The introduction port 111 may be arranged along the perimeter of the base 114. The side surface of the base 114 may have a grill shape to facilitate the entry and exit of air.

The grill assembly 120 may be provided on an upper portion of the housing body 113. The grill assembly 120 may be arranged above a fan 130 to be described later. The grill assembly 120 may form the upper surface of the air purifier 10. The grill assembly 120 may be formed to cover the internal space of the housing 110 from above.

The grill assembly 120 may have a grill shape to allow air to enter and exit through the discharge port 112. The grill shape may be arranged along the perimeter of the upper surface of the housing 110. However, the arrangement of the grill shape is not limited thereto and may vary.

The body frame 150 may be arranged inside the housing 110. The body frame 150 may be configured to support the grill assembly 120. The body frame 150 may be coupled to the inner surface of the housing 110. The body frame 150 may support the inner surface of the housing 110.

The body frame 150 may be provided to support various parts of the air purifier 10. The parts of the air purifier 10, such as a filter assembly 160 and a fan 130, may be supported by the body frame 150.

The body frame 150 may be covered by the housing 110. For example, the side of the body frame 150 in the horizontal direction may be covered by the housing body 113. The bottom of the body frame 150 in the vertical direction may be covered by the base 114. The top of the body frame 150 in the vertical direction may be covered by the grill assembly 120.

The body frame 150 may be formed to be divided into a plurality of frames. For example, the body frame 150 may include an inner frame 151 and a cover frame 152 covering the side of the inner frame 151.

The inner frame 151 may be provided to support various parts of the air purifier 10. For example, a power supply unit of the air purifier 10 may be supported on the outer surface of the inner frame 151. A space having a substantially cylindrical shape may be formed inside the inner frame 151.

The filter assembly 160, the fan 130, and the like may be arranged in the internal space of the inner frame 151. The inner frame 151 may have an open surface 1501 that is open to allow the filter assembly 160 to be inserted or removed. Through the open surface 1501, a user may easily insert or remove the filter assembly 160. The first flow path P1 may be provided in the internal space of the inner frame 151. The inner frame 151 may cover an outer side of the first flow path P1.

The cover frame 152 may cover the side of the inner frame 151. A plurality of cover frames 152 may be provided. The cover frame 152 may prevent a configuration arranged on the outer surface of the inner frame 151 from being exposed to the outside.

However, the shape of the body frame 150 is not limited thereto and the body frame 150 may have various shapes. For example, the space formed inside the body frame 150 may have a shape other than the cylindrical shape.

A plurality of frames constituting the body frame 150 may be formed to form an integral body frame 150 while being coupled to each other. Accordingly, the body frame 150 may be easily disassembled or assembled. However, the structure of the body frame 150 is not limited thereto and may be formed as an integrally formed single configuration.

The air purifier 10 according to an embodiment may include the fan 130 arranged inside the housing 110. The fan 130 may be provided in the first flow path P1. The fan 130 may be arranged between the introduction port 111 and the discharge port 112. As the fan 130 rotates, an air flow may be generated in a direction from the introduction port 111 toward the discharge port 112. Accordingly, external air of the housing 110 may be introduced through the introduction port 111, and the introduced air may move along the first flow path P1 to be discharged through the discharge port 112.

The fan 130 may be configured as an axial-flow type. The fan 130 may include a blade 131, a motor 132 that supplies power to the blade 131, and a fan rotation shaft 133 that is connected to the blade 131 and the motor 132 to transmit power generated by the motor 132 to the blade 131. However, the type and configuration of the fan 130 are not limited thereto and may be modified as needed. For example, the fan 130 may be configured as a centrifugal type.

The fan 130 may be arranged between the filter assembly 160 and the discharge port 112. When the fan 130 is driven, air introduced into the introduction port 111 may sequentially pass through the filter assembly 160 and the fan 130 and be then discharged through the discharge port 112. However, the arrangement of the fan 130 is not limited thereto and may vary. For example, unlike the drawings, the fan 130 may be arranged between the introduction port 111 and the filter assembly 160.

The filter assembly 160 may remove foreign substances from the air introduced into the housing 110. The filter assembly 160 may be provided in the first flow path P1. When the fan 130 is driven, the air introduced through the introduction port 111 may move along the first flow path P1 and pass through the filter assembly 160. Foreign substances in the air moving along the first flow path P1 may be removed by the filter assembly 160. The air from which the foreign substances have been removed by the filter assembly 160 may be discharged through the discharge port 112.

The filter assembly 160 may have a shape corresponding to the shape of the housing 110. For example, the filter assembly 160 may have a cylindrical structure. However, the shape of the filter assembly 160 is not limited thereto and may vary depending on the shape of the housing 110.

The filter assembly 160 may include a dust collection filter 161 and a pre-filter 162.

The pre-filter 162 may be provided in the first flow path P1. The pre-filter 162 may be arranged on a flow path that is toward the dust collection filter 161. The pre-filter 162 may be arranged between the introduction port 111 and the dust collection filter 161, in the first flow path P1.

The pre-filter 162 may be provided to primarily collect foreign substances in air sucked in through the introduction port 111. The pre-filter 162 may be provided to separate relatively large foreign substances from air. For example, the pre-filter 162 may be configured to filter out foreign substances of a first size.

The dust collection filter 161 may be provided in the first flow path P1. The dust collection filter 161 may be arranged downstream of the pre-filter 162 in the first flow path P1. The dust collection filter 161 may be provided to separate foreign substances that are smaller than foreign substances separated by the pre-filter 162. For example, the dust collection filter 161 may be configured to filter out foreign substances of a second size smaller than the first size.

The dust collection filter 161 may include an electric dust collection filter configured to collect dust by using electrostatic force. When power is applied to the electric dust collection filter, an electric field may be formed in the electric dust collection filter. Foreign substances in air, such as dust, may be collected by the electric field formed in the electric dust collection filter. In addition, organic substances, such as bacteria or viruses may be decomposed by the electric field formed in the electric dust collection filter. For example, the electric dust collection filter may perform dust collection and sterilization functions.

The electric dust collection filter may include a charging unit 163 and a dust collection unit 164.

The charging unit 163 may be arranged to charge foreign substances in air. The charging unit 163 may be provided above the pre-filter 162 to charge foreign substances in air that has passed through the pre-filter 162. In other words, the charging unit 163 may be arranged downstream of the pre-filter 162 in the first flow path P1.

The dust collection unit 164 may be provided to collect the foreign substances charged by the charging unit 163. Electrodes having different polarities may be connected to the charging unit 163 and the dust collection unit 164, respectively. An electric field may be formed between the charging unit 163 and the dust collection unit 164. The foreign substances in the air that are charged by the charging unit 163 may be charged with the same polarity as that of the charging unit 163 and may be charged with the opposite polarity to that of the dust collection unit 164. Accordingly, the foreign substances in the air that are charged by the charging unit 163 may be moved to the dust collection unit 164 by electric force and then collected by the dust collection unit 164. In addition, organic substances, such as bacteria or viruses floating in the air may be decomposed by the electric force.

The dust collection unit 164 may be provided on one side of the electric dust collection filter, and the dust collection unit 164 may be provided on the other side of the electric dust collection filter. The charging unit 163 and the dust collection unit 164 may be arranged to face each other. For example, the charging unit 163 and the dust collection unit 164 may be arranged to face each other in the vertical direction. The dust collection unit 164 may be provided above the electric dust collection filter, and the charging unit 163 may be provided below the electric dust collection filter. The dust collection unit 164 may be arranged downstream of the charging unit 163 in the first flow path P1. The charging unit 163 may be arranged between the dust collection unit 164 and the introduction port 111. The dust collection unit 164 may be arranged between the charging unit 163 and the discharge port 112.

The pre-filter 162 may be arranged between the introduction port 111 and the electric dust collection filter. In other words, the electric dust collection filter may be arranged between the discharge port 112 and the pre-filter 162.

The above-described embodiment of the disclosure, an example is mainly described in which the dust collection filter 161 includes the electric dust collection filter, but the disclosure is not limited thereto, and the dust collection filter 161 may further include another dust collection filter or may include another dust collection filter instead. For example, the dust collection filter 161 may include various filters, such as a high-efficiency particulate air (HEPA) filter.

The air purifier 10 may include a control unit 171 for controlling the operation of the air purifier 10. The control unit 171 may include a first printed circuit board assembly 172. The first printed circuit board assembly 172 may be configured to control the operation of the air purifier 10. The first printed circuit board assembly 172 may be formed by mounting electronic components for controlling the operation of the air purifier 10 on a printed circuit board. The control unit 171 may be electrically connected to various parts, such as the fan 130 and the electric dust collection filter.

Meanwhile, the air purifier 10 according to an embodiment may have a structure that is easy to assemble. For example, the air purifier 10 may have a structure that may simplify the assembly process by reducing the use of fastening members, such as screws.

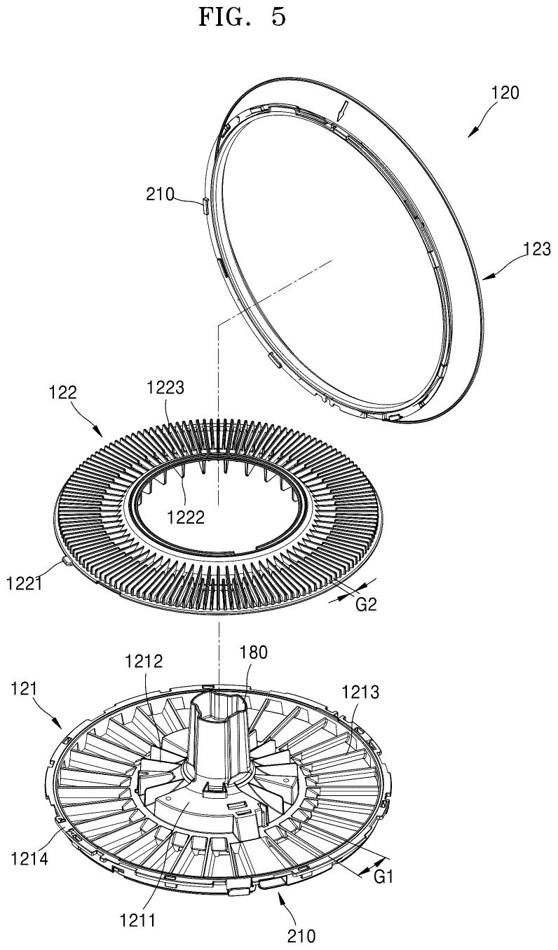

FIG. 5 is an exploded perspective view illustrating a configuration of a grill assembly 120 according to an embodiment of the disclosure.

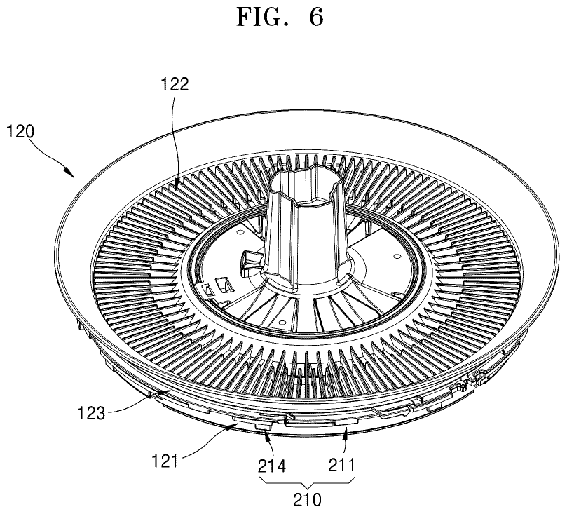

FIG. 6 is an assembled perspective view illustrating a configuration of the grill assembly 120 according to an embodiment of the disclosure.

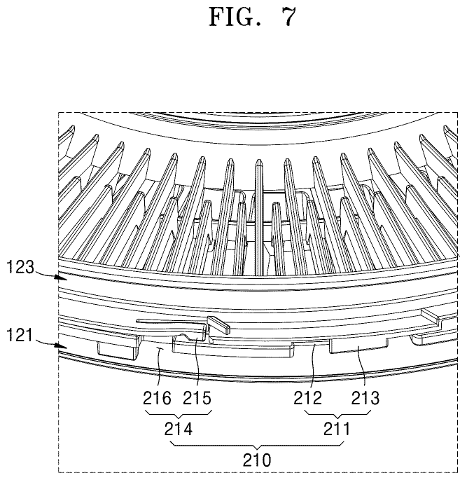

FIG. 7 is a perspective view illustrating a state before assembly of a first grill frame and an edge frame of a grill assembly according to an embodiment of the disclosure.

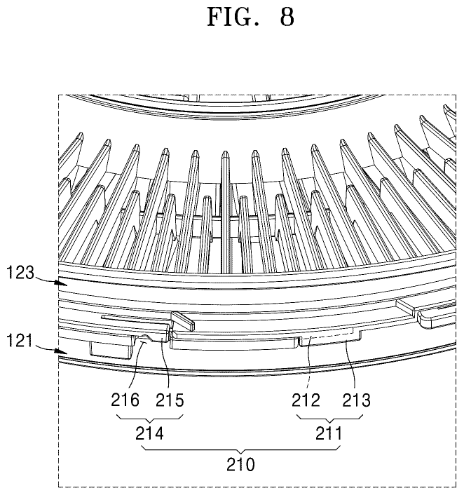

FIG. 8 is a perspective view illustrating a state after assembly of a first grill frame and an edge frame of a grill assembly according to an embodiment of the disclosure.

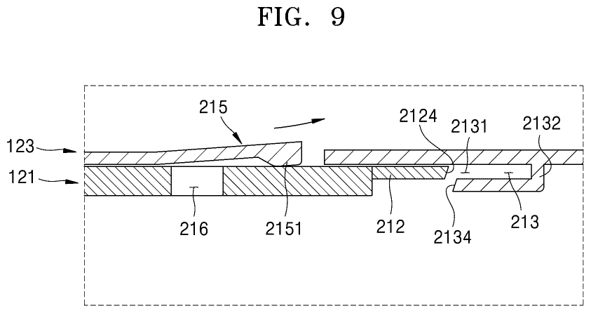

FIG. 9 is a cross-sectional view conceptually illustrating a state before assembly of a first grill frame and an edge frame of FIG. 7 according to an embodiment of the disclosure.

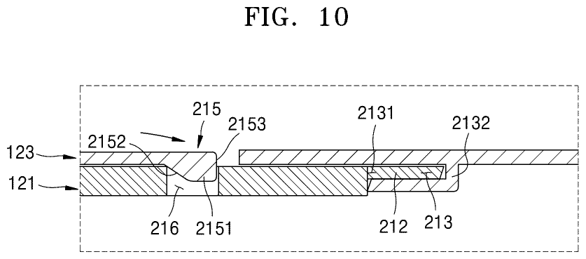

FIG. 10 is a cross-sectional view conceptually illustrating a state after assembly of a first grill frame and an edge frame of FIG. 8 according to an embodiment of the disclosure.

Referring to FIGS. 2, 5, and 6, the grill assembly 120 according to an embodiment may form the upper exterior of the air purifier 10. The grill assembly 120 may include the first grill frame 121, a second grill frame 122, and the edge frame 123.

The first grill frame 121 may have a grill shape to allow air to enter and exit through the discharge port 112. For example, the first grill frame 121 may have first grills 1213 arranged at first intervals G1. The first grills 1213 may be provided along the outer edge of a central portion 1211. In other words, the first grills 1213 may be arranged in an edge area of the first grill frame 121.

The first grills 1213 may cover a periphery portion at an upper portion of the housing body 113. The first grills 1213 may have a perforated shape to allow air to flow. The first grills 1213 may be arranged to be inclined with respect to the vertical direction by considering a direction of air discharged through the discharge port 112. In order for the first grills 1213 to be formed to be inclined with respect to the vertical direction, injection molding may be used. Based on a mold for injection molding, the first intervals G1 may be a predetermined size or greater. For example, the first intervals may be 10 millimeters (mm) or greater. The first intervals G1 may be less than 50 mm.

The central portion 1211 of the first grill frame 121 may include a guide portion 1212 extending from the first grill 1213 toward a connection flow path. The guide portion 1212 may be provided to guide air from the first grill 1213 to the connection flow path. The guide portion 1212 may be formed along the perimeter of the central portion 1211. The guide portion 1212 may be partitioned by a plurality of rib structures.

A first unit coupling portion 180 for coupling with an air sterilizer 30 may be arranged in the central portion 1211. The first unit coupling portion 180 may extend upward from the central portion 1211. For example, the guide portion 1212 may be arranged along the circumferential direction of the first unit coupling portion 180.

The second grill frame 122 may be arranged above the first grill frame 121. The second grill frame 122 may be arranged above the first grill 1213 of the first grill frame 121. The second grill frame 122 may perform a function of blocking a user's hand from entering through the first grill 1213 of the first grill frame 121.

As an example for this, the second grill frame 122 may have second grills 1223 arranged at second intervals G2 smaller than the first intervals G1. The second intervals G2 may be less than ½ of the first intervals G1. The second intervals G2 may be intervals that may prevent a user's finger from being inserted. For example, the second intervals G2 may be less than 10 mm. For example, the second intervals G2 may be less than 7 mm. For example, the second intervals G2 may be less than 5 mm.

The inclination angle of the second grill 1223 with respect to the vertical direction may be less than the inclination angle of the first grill 1213 with respect to the vertical direction. For example, when the inclination angle of the first grill 1213 with respect to the vertical direction is 10 degrees or more, the inclination angle of the second grill 1223 with respect to the vertical direction may be less than 10 degrees.

In this way, the second grills 1223 having the second intervals G2 may be arranged more closely than the first grills 1213 having the first intervals G1, thereby preventing a user's hand from entering the inside of the housing body 113 and thus preventing the user's hand from touching the fan 130.

The second grill frame 122 may include an inner edge arranged on the inside of the second grill 1223 and an outer edge arranged on the outside of the second grill 1223. The inner edge may have a structure that protrudes upward more than the second grill 1223. The second grill frame 122 may be provided with a plurality of spacers 1222 that protrude toward the first grill frame 121. The plurality of spacers 1222 may be arranged below the inner edge and may be spaced apart from each other in the circumferential direction. When the second grill frame 122 is arranged on the first grill frame 121, the spacers 1222 may maintain a constant vertical gap between the second grill frame 122 and the first grill frame 121. Therefore, a predetermined space may be secured for air passing through the first grill 1213 of the first grill frame 121 to move along the guide portion 1212.

A rotation prevention member 1221 may be arranged on the outer edge of the second grill frame 122. For example, the rotation prevention member 1221 may be in the form of a protrusion protruding in a radial direction. The rotation prevention member 1221 of the second grill frame 122 may be seated on a rotation prevention member 1214 formed on the outer edge of the first grill frame 121. In the process of arranging the second grill frame 122 on the first grill frame 121, the rotation prevention member 1221 of the second grill frame 122 may be arranged to be seated on the rotation prevention member 1214 of the first grill frame 121.

The edge frame 123 may be arranged on the second grill frame 122. The edge frame 123 may be arranged to overlap the outer edge of the second grill frame 122. The inner diameter of the edge frame 123 may be greater than the outer diameter of the second grill frame 122.

The edge of the second grill frame 122 may be arranged between the first grill frame 121 and the edge frame 123. Accordingly, the second grill frame 122 may be fixed in the vertical direction by the first grill frame 121 and the edge frame 123.

The edge frame 123 may be detachably assembled to the first grill frame 121. The edge frame 123 may be rotational assembled to be fixed to the first grill frame 121 without a fastening member.

For example, after the edge frame 123 and the first grill frame 121 are brought closer together in the vertical direction, the edge frame 123 may be assembled onto the first grill frame 121 by rotating the edge frame 123 in a predetermined rotation assembly direction, for example, clockwise, with respect to the first grill frame 121.

The assembled edge frame 123 may be fixed in the vertical direction with respect to the first grill frame 121 and may be assembled such that rotational movement is restricted with respect to the first grill frame 121. As an example for this, the grill assembly 120 may include a first rotation assembly structure 210 configured to rotationally assemble the edge frame 123 onto the first grill frame 121 such that the edge frame 123 is fixed in the vertical direction with respect to the first grill frame 121 and the rotational movement is restricted with respect to the first grill frame 121. For example, at least one of the edge frame 123 and the first grill frame 121 may include the first rotation assembly structure 210. For example, the edge frame 123 and the first grill frame 121 may each include the first rotation assembly structure 210.

Referring to FIGS. 7 and 8, the first rotation assembly structure 210 may be provided on the outer surface of each of the edge frame 123 and the first grill frame 121. By providing the first rotation assembly structure 210 on the outer surface of each of the edge frame 123 and the first grill frame 121, a user or worker may easily check an assembly structure of the edge frame 123 and the first grill frame 121 with the naked eye. Therefore, the first rotation assembly structure 210 may be easily assembled or disassembled without damage to the first rotation assembly structure 210.

The first rotation assembly structure 210 may include a first separation prevention structure 211 for preventing the edge frame 123 from being separated in the vertical direction and a first rotation prevention structure 214 for preventing the edge frame 123 from being rotated.

The first separation prevention structure 211 may include a first rotation-inducing protrusion 212 provided on one of the first grill frame 121 and the edge frame 123, and a first rotation-inducing groove 213 provided on the other of the first grill frame 121 and the edge frame 123.

For example, a plurality of first rotation-inducing protrusions 212 may be provided on the outer surface of the first grill frame 121, and a plurality of first rotation-inducing grooves 213 may be provided on the outer surface of the edge frame 123. The first rotation-inducing groove 213 may allow rotational movement of the first rotation-inducing protrusion 212, but may restrict movement in the vertical direction perpendicular to the rotational movement.

Referring to FIGS. 9 and 10, the first rotation-inducing groove 213 may extend in the circumferential direction. One end 2131 of the first rotation-inducing groove 213 in the circumferential direction may be opened to allow insertion of the first rotation-inducing protrusion 212, and the other end 2132 of the first rotation-inducing groove 213 in the circumferential direction may have a structure in which the first rotation-inducing protrusion 212 is closed to restrict movement.

Accordingly, while the first rotation-inducing protrusion 212 is inserted through one end 2131 of the first rotation-inducing groove 213, the edge frame 123 may be rotated at a predetermined rotation angle and in a rotation assembly direction.

An inclined structure may be formed in at least one of the first rotation-inducing protrusion 212 and the first rotation-inducing groove 213 such that the first rotation-inducing protrusion 212 may be easily inserted into the first rotation-inducing groove 213. For example, a first inclined surface 2134 inclined with respect to the rotation assembly direction may be formed on the open end 2131 of the first rotation-inducing groove 213. A second inclined surface 2124 facing the first inclined surface 2134 may be formed on the first rotation-inducing protrusion 212.

Through the first inclined surface 2134 and the second inclined surface 2124, when the first rotation-inducing protrusion 212 is inserted into or separated from the first rotation-inducing groove 213, natural movement is possible without damage to the first rotation-inducing protrusion 212 or the first rotation-inducing groove 213.

The first rotation prevention structure 214 may include a first hook 215 provided on one of the first grill frame 121 and the edge frame 123, and a first hook-catching portion 216 provided on the other of the first grill frame 121 and the edge frame 123.

For example, the first hook 215 may be provided on the outer surface of the edge frame 123, and the first hook-catching portion 216 may be provided on the outer surface of the first grill frame 121.

The first hook 215 may be arranged adjacent to the first rotation-inducing groove 213 on the outer side of the edge frame 123. The first hook 215 may be spaced apart from the first rotation-inducing groove 213 in the edge frame 123 in the circumferential direction.

The first hook 215 may have a free end that is elastically deformable. The first hook 215 may be elastically deformable in a direction inclined with respect to the rotation assembly direction. The first hook 215 may include a first protrusion 2151 that protrudes toward the first grill frame 121. The first protrusion 2151 may protrude downward.

Referring to FIG. 10, when the edge frame 123 is rotated at a predetermined angle, the first hook-catching portion 216 may be provided such that the first hook 215 is inserted thereinto. When the first hook 215 is inserted into the first hook-catching portion 216, the first hook 215 may be restricted from moving in the rotation assembly direction by the first hook-catching portion 216.

For example, because the end of the first hook 215 is caught by the first hook-catching portion 216, the rotation may be restricted in a counterclockwise direction. For example, the first hook 215 may be configured such that the edge frame 123 may rotate in the rotation assembly direction, for example, clockwise, and the rotation is restricted in a direction opposite to the rotation assembly direction, for example, counterclockwise.

For example, the first protrusion 2151 of the first hook 215 may provide a first surface 2152 that is inclined with respect to the rotation assembly direction, and a second surface 2153 that is perpendicular to the rotation assembly direction. Accordingly, the second surface 2153 of the first hook 215 may be caught by the first hook-catching portion 216, and thus, the rotation in the counterclockwise direction may be restricted.

Meanwhile, because an end of the first rotation-inducing protrusion 212 contacts the other end 2132 of the first rotation-inducing groove 213 and the movement of the edge frame 123 is restricted, the rotation in the clockwise direction may be restricted. Accordingly, unless a user intentionally lifts the first hook 215, the edge frame 123 and the first grill frame 121 are restricted from moving in the vertical direction and rotating.

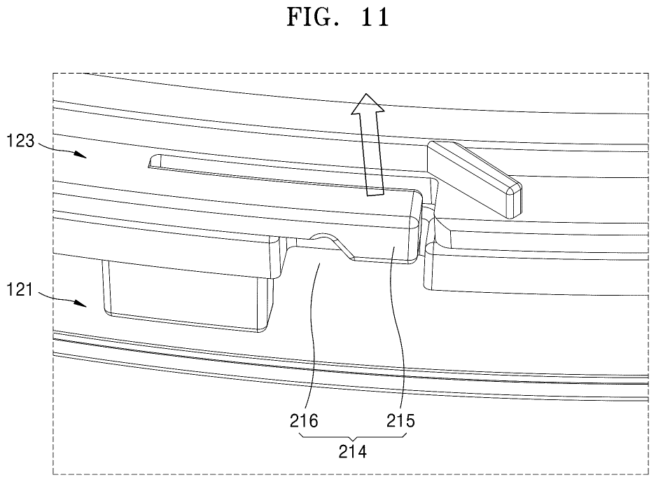

FIG. 11 is a diagram illustrating an operation for separating an edge frame from a first grill frame in a grill assembly according to an embodiment of the disclosure.

Referring to FIG. 11, when a user or worker wants to separate the edge frame 123, the edge frame 123 may be separated from the first grill frame 121 by intentionally lifting the first hook 215 and rotating the edge frame 123 in a direction opposite to the rotation assembly direction, for example, counterclockwise.

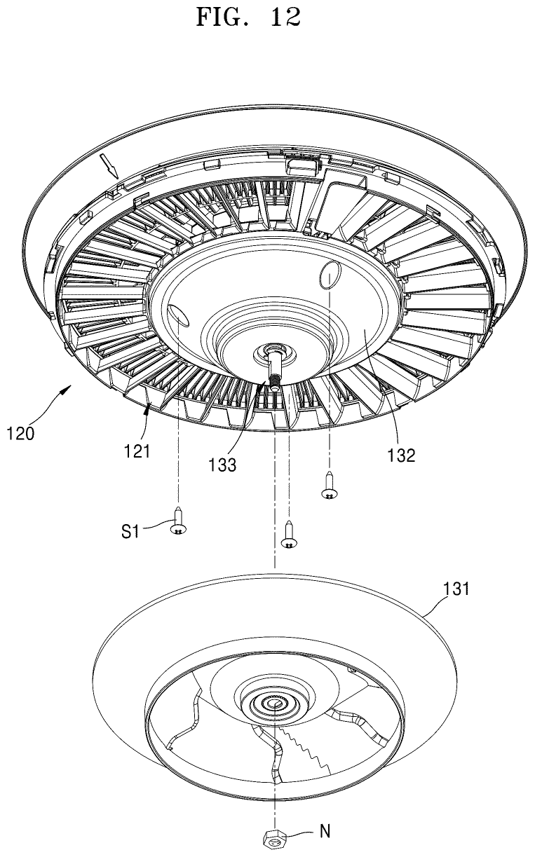

FIG. 12 is an exploded perspective view illustrating a structure for assembling a fan to a grill assembly according to an embodiment of the disclosure.

Referring to FIG. 12, a fan 130 may be fixed to a lower portion of the grill assembly 120 according to an embodiment. The fan 130 may be fixed to the grill assembly 120 such that a fan rotation shaft 133 is located at the center of the grill assembly 120.

The fan 130 may include a blade 131, a motor 132 that provides rotational power to the blade 131, and the fan rotation shaft 133 that transmits the rotational power of the motor 132 to the blade. For example, the motor 132 may be fixed to a central portion 1211 of the first grill frame 121 of the grill assembly 120. The motor 132 may have a plurality of fastening holes. As a fastening member S1 is coupled to the fastening holes, the motor 132 may be fixed to the first grill frame 121 of the grill assembly 120.

The blade 131 may be screw-coupled to the fan rotation shaft 133. The fan rotation shaft 133 passes through the central portion 1211 of the blade 131, and a screw N may be coupled to the end of the fan rotation shaft 133. Accordingly, the blade 131 may be fixed to the fan rotation shaft 133.

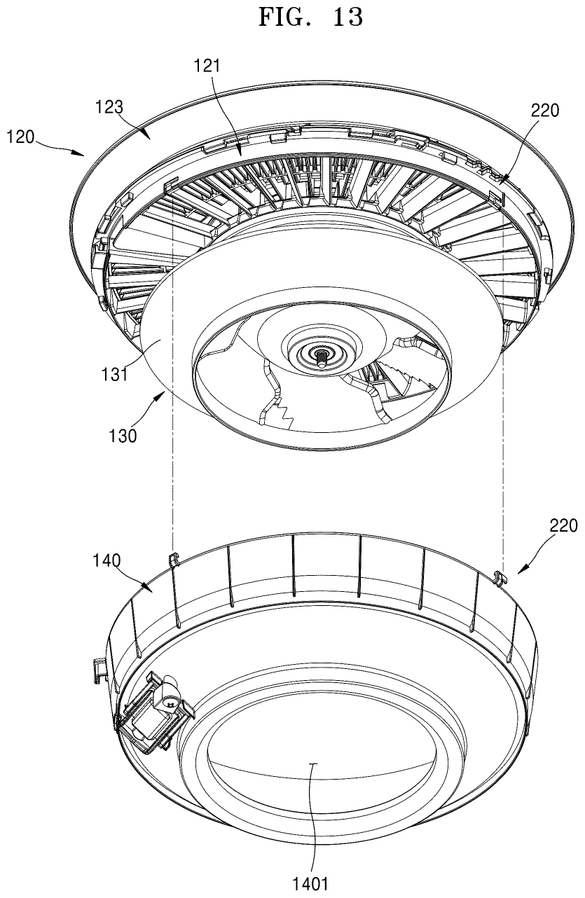

FIG. 13 is an exploded perspective view illustrating a structure of assembling a duct frame to a grill assembly of FIG. 12 according to an embodiment of the disclosure.

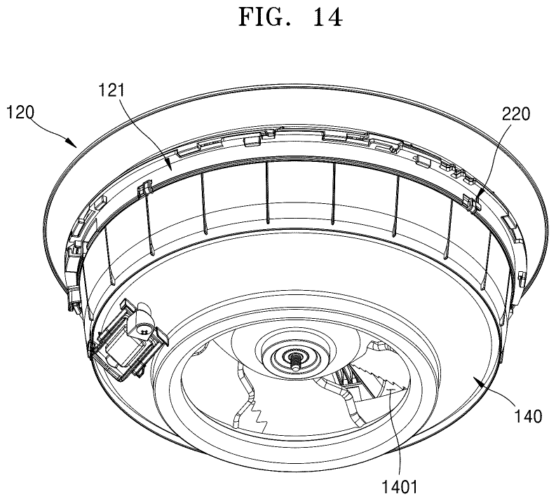

FIG. 14 is an assembled perspective view illustrating a structure of assembling a duct frame to a grill assembly onto which a fan of FIG. 12 is assembled according to an embodiment of the disclosure.

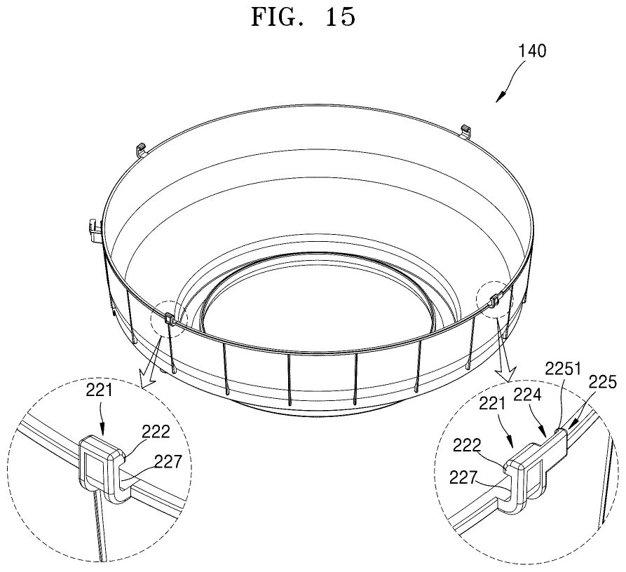

FIG. 15 is a perspective view illustrating a duct frame according to an embodiment of FIG. 13 according to an embodiment of the disclosure.

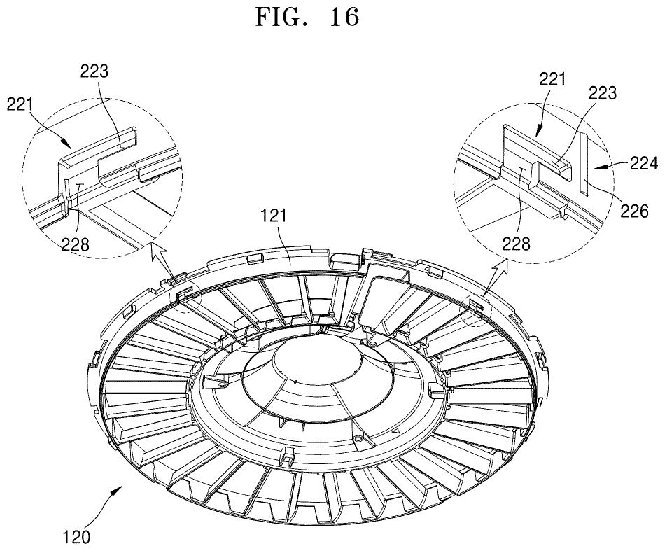

FIG. 16 is a perspective view illustrating a first grill frame of a grill assembly according to the embodiment of FIG. 13 according to an embodiment of the disclosure.

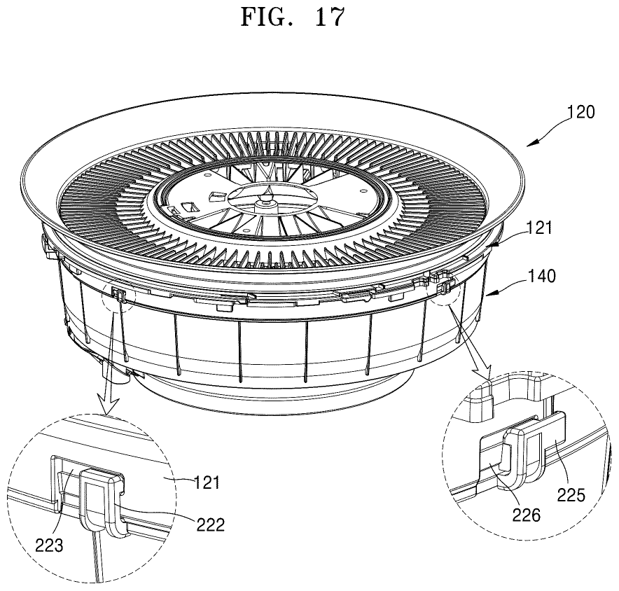

FIG. 17 is a diagram illustrating an assembly structure of a grill assembly and a duct frame according to an embodiment of the disclosure.

Referring to FIGS. 13 and 14, in the grill assembly 120 according to an embodiment of the disclosure, the fan 130 is installed in a lower portion of the grill assembly 120, and the duct frame 140 may be arranged to surround the fan.

The duct frame 140 may be arranged to accommodate the fan 130 inside the housing body 113. The duct frame 140 may include an intake port 1401 arranged on the first flow path P1. As the fan 130 arranged inside the duct frame 140 operates, air drawn in through the introduction port 111 is sucked into the intake port 1401, and the sucked air is discharged through the discharge port 112. The diameter of the intake port 1401 is less than the diameter of the housing body 113.

The duct frame 140 may be detachably assembled onto the outer surface of the grill assembly 120. The grill assembly 120 may be rotational assembled to be fixed to the duct frame 140 without a fastening member.

For example, after the grill assembly 120 and the duct frame 140 are brought closer together in the vertical direction, the grill assembly 120 may be assembled onto the duct frame 140 by rotating the grill assembly 120 in a predetermined rotation assembly direction, for example, clockwise, with respect to the duct frame 140.

The assembled grill assembly 120 may be fixed in the vertical direction with respect to the duct frame 140 and may be assembled such that rotational movement is restricted with respect to the duct frame 140. As an example for this, at least one of the grill assembly 120 and the duct frame 140 may include a second rotation assembly structure 220 configured to rotationally assemble the grill assembly 120 onto the duct frame 140 such that the grill assembly 120 is fixed in the vertical direction with respect to the duct frame 140 and the rotational movement is restricted with respect to the duct frame 140. For example, the grill assembly 120 and the duct frame 140 may include the second rotation assembly structure 220.

Referring to FIGS. 15 and 16, the second rotation assembly structure 220 may be provided on the outer surface of each of the duct frame 140 and the grill assembly 120. For example, the second rotation assembly structure 220 may be provided on the outer surface of each of the duct frame 140 and the first grill frame 121. Through this, a user or worker may easily check an assembly structure of the duct frame 140 and the first grill frame 121 with the naked eye. Therefore, the second rotation assembly structure 220 may be easily assembled or disassembled without damage to the second rotation assembly structure 220.

A rotation assembly direction of the second rotation assembly structure 220 may be the same as a rotation assembly direction of the first rotation assembly structure 210. In other words, the rotation assembly direction of the first rotation assembly structure 210 may be the same as the rotation assembly direction of the second rotation assembly structure 220. By designing the rotation assembly directions of the first rotation assembly structure 210 and the second rotation assembly structure 220 to be the same, the already assembled first rotation assembly structure 210 may be prevented from being separated during a rotation assembly process of the second rotation assembly structure 220.

The second rotation assembly structure 220 may include a second separation prevention structure 221 for preventing separation in the vertical direction and a second rotation prevention structure 224 for preventing rotation in the rotation direction.

The second separation prevention structure 221 may include a second rotation-inducing protrusion 222 provided on one of the first grill frame 121 and the duct frame 140, and a second rotation-inducing groove 223 provided on the other of the first grill frame 121 and the duct frame 140.

For example, a plurality of second rotation-inducing protrusions 222 may be provided on the outer surface of the duct frame 140, and a plurality of second rotation-inducing grooves 223 into which the second rotation-inducing protrusions 222 may be inserted may be provided on the outer surface of the first grill frame 121.

The duct frame 140 may include a plurality of protrusion support portions 227 extending upwardly, and a second rotation-inducing protrusion 222 provided on the inner surface of each of the plurality of protrusion support portions 227. The second rotation-inducing protrusion 222 may have a structure protruding inwardly from the inner surface of the protrusion support portion 227.

The second rotation-inducing groove 223 of the first grill frame 121 may be connected to a guide groove 228 in which the second rotation-inducing protrusion 222 may move in the vertical direction. The second rotation-inducing groove 223 may extend in a rotational direction with respect to the guide groove 228.

The second rotation-inducing groove 223 may have a structure in which one end, for example, the left end, is open. One end of the second rotation-inducing groove 223 may be connected to the guide groove 228. Accordingly, the second rotation-inducing protrusion 222 may be inserted through one end of the second rotation-inducing groove 223 after moving in the vertical direction. The second rotation-inducing groove 223 may have a structure in which the other end, for example, the right end, is closed. Accordingly, the second rotation-inducing protrusion 222 inserted through one end of the second rotation-inducing groove 223 may contact and be supported by the other end of the second rotation-inducing groove 223.

The second rotation prevention structure 224 may include a second hook 225 provided on one of the first grill frame 121 and the duct frame 140, and a second hook-catching portion 226 provided on the other of the first grill frame 121 and the duct frame 140. For example, the second hook-catching portion 226 may be provided on the outer surface of the first grill frame 121, and the second hook 225 may be provided on the outer surface of the duct frame 140.

For example, the second hook 225 may be provided on at least some of the plurality of protrusion support portions 227. For example, the second hook 225 may have a structure extending in the circumferential direction and may have a free end that is elastically deformable. The second hook 225 may be elastically deformed in a direction inclined with respect to the circumferential direction. The second hook 225 may include a second protrusion 2251 protruding toward the first grill frame 121. For example, the second protrusion 2251 may protrude inwardly.

The second hook-catching portion 226 may be provided on the side of the second rotation-inducing groove 223 in the circumferential direction. The second hook-catching portion 226 may be arranged to be spaced apart from the second rotation-inducing groove 223 in the circumferential direction on the outer surface of the first grill frame 121.

Referring to FIG. 17, when the grill assembly 120 is moved in the vertical direction on the duct frame 140, and then the grill assembly 120 or the duct frame 140 is rotated, the second rotation-inducing protrusion 222 is inserted into the second rotation-inducing groove 223. When the grill assembly 120 is rotated by a predetermined angle, the second hook 225 may be caught on the second hook-catching portion 226. Accordingly, the duct frame 140 and the grill assembly 120 may be restricted from mutual rotational movement and up and down movement.

When a user wants to separate the grill assembly 120 from the duct frame 140, the second hook 225 of the second rotation assembly structure 220 exposed to the outside may be separated from the second hook-catching portion 226 to rotate the grill assembly 120 or the duct frame 140 and move the grill assembly 120 or the duct frame 140 up and down.

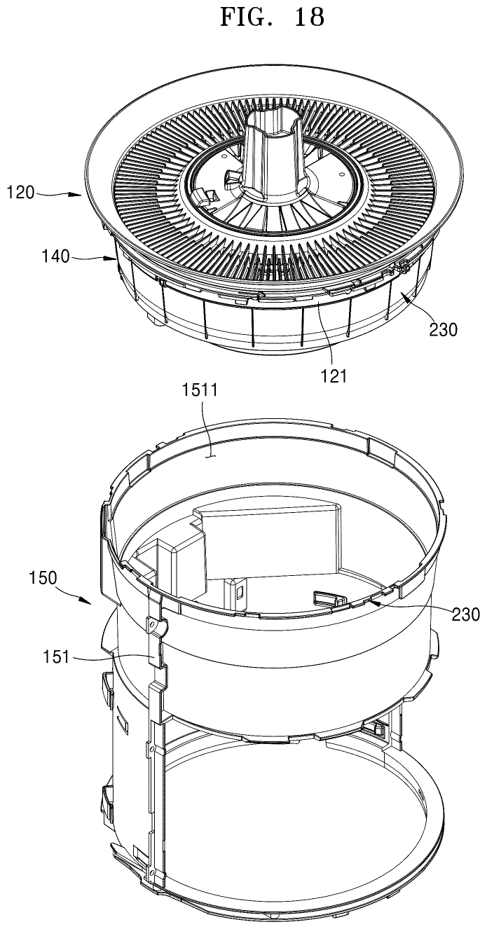

FIG. 18 is an exploded perspective view illustrating a state before the grill assembly 120 is assembled onto the body frame 150 according to an embodiment of the disclosure.

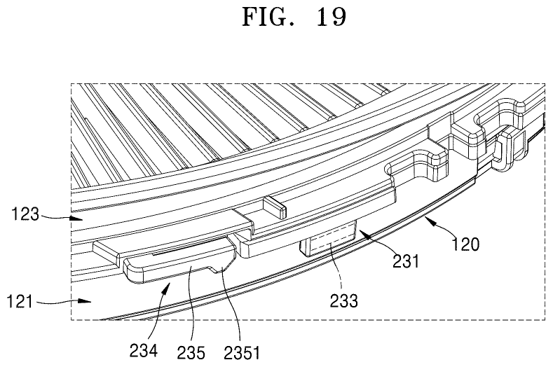

FIG. 19 is an enlarged perspective view illustrating a portion of the grill assembly 120 in FIG. 18 according to an embodiment of the disclosure.

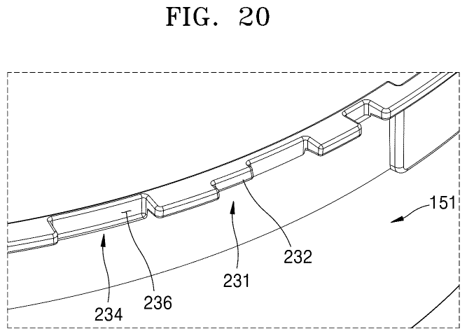

FIG. 20 is an enlarged perspective view illustrating a portion of the body frame 150 in FIG. 18 according to an embodiment of the disclosure.

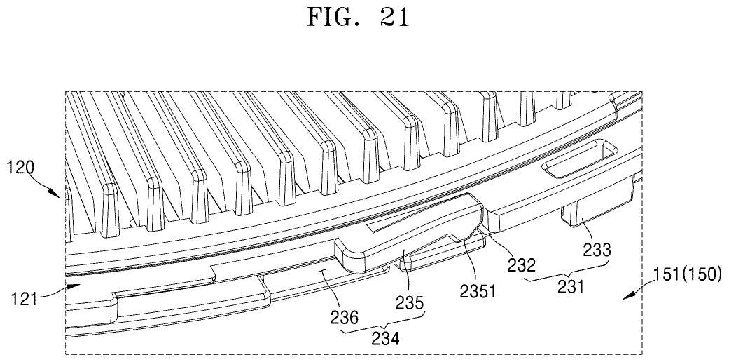

FIG. 21 illustrates a state in which the grill assembly 120 of FIG. 18 is seated on the inner frame 151 according to an embodiment of the disclosure.

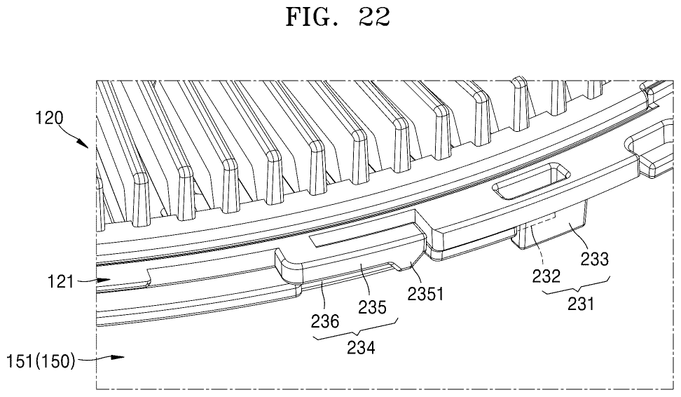

FIG. 22 illustrates a state in which the grill assembly 120 of FIG. 21 is assembled onto the inner frame according to an embodiment of the disclosure.

Referring to FIG. 4 and FIGS. 18 to 22, in the air purifier 10 according to an embodiment of the disclosure, the grill assembly 120 may be supported on the inner frame 151 of the body frame 150.

The grill assembly 120 may be supported on an upper portion of the inner frame 151. The inner frame 151 may have an opening 1511 into which a duct frame 140 may be inserted. The duct frame 140 is arranged inside the inner frame 151 through the opening 1511, and accordingly, a first flow path P1 moving toward the fan 130 may be formed inside the inner frame 151. The inner frame 151 may be formed as an integrally formed single configuration.

The grill assembly 120 may be detachably assembled onto the inner frame 151. The grill assembly 120 may be rotational assembled to be fixed to the inner frame 151 without a fastening member.

For example, after the grill assembly 120 and the inner frame 151 are brought closer together in the vertical direction, the grill assembly 120 may be assembled onto the inner frame 151 by rotating the grill assembly 120 in a predetermined rotation assembly direction, for example, clockwise, with respect to the inner frame 151.

The assembled grill assembly 120 may be fixed in the vertical direction with respect to the inner frame 151 and may be assembled such that rotational movement is restricted with respect to the inner frame 151. As an example for this, at least one of the grill assembly 120 and the body frame 150 may have a third rotation assembly structure 230 configured to rotationally assemble the grill assembly 120 onto the body frame 150 such that the grill assembly 120 is fixed in the vertical direction with respect to the body frame 150 and the rotational movement is restricted with respect to the body frame 150. For example, the grill assembly 120 and the inner frame 151 may each include a third rotation assembly structure 230.

The third rotation assembly structure 230 may be provided on the outer surface of each of the grill assembly 120 and the inner frame 151. By providing the third rotation assembly structure 230 on the outer surface of each of the grill assembly 120 and the inner frame 151, a user or worker may easily check an assembly structure of the grill assembly 120 and the inner frame 151. Therefore, the third rotation assembly structure 230 may be easily assembled or disassembled without damage to the third rotation assembly structure 230.

A rotation assembly direction of the third rotation assembly structure 230 may be the same as a rotation assembly direction of the first rotation assembly structure 210. In other words, the rotation assembly direction of the first rotation assembly structure 210 may be the same as the rotation assembly direction of the third rotation assembly structure 230. By designing the rotation assembly directions of the first rotation assembly structure 210 and the third rotation assembly structure 230 to be the same, the already assembled first rotation assembly structure 210 may be prevented from being separated during a rotation assembly process of the third rotation assembly structure 230.

The third rotation assembly structure 230 may include a third separation prevention structure 231 for preventing the grill assembly 120 from being separated in the vertical direction and a third rotation prevention structure 234 for preventing the grill assembly 120 from being rotated.

The third separation prevention structure 231 may include a third rotation-inducing protrusion 232 provided on one of the first grill frame 121 and the inner frame 151, and a third rotation-inducing groove 233 provided on the other of the first grill frame 121 and the inner frame 151.

For example, the third rotation-inducing protrusion 232 may be provided on the outer surface of the inner frame 151, and the third rotation-inducing groove 233 may be provided on the outer surface of the first grill frame 121. The third rotation-inducing groove 233 may allow rotational movement of the third rotation-inducing protrusion 232, but may restrict movement in the vertical direction perpendicular to the rotational movement.

The third rotation-inducing groove 233 may extend in a rotational direction. One end of the third rotation-inducing groove 233 in the rotational direction may be opened to allow insertion of the third rotation-inducing protrusion 232, and the other end of the third rotation-inducing groove 233 in the rotational direction may have a structure in which the third rotation-inducing protrusion 232 is closed to restrict movement.

Accordingly, while the third rotation-inducing protrusion 232 is inserted through one end of the third rotation-inducing groove 233, the grill assembly 120 may be rotated at a predetermined rotation angle and in a rotation assembly direction An inclined structure may be formed in at least one of the third rotation-inducing protrusion 232 and the third rotation-inducing groove 233 such that the third rotation-inducing protrusion 232 may be easily inserted into the third rotation-inducing groove 233.

The third rotation prevention structure 234 may include a third hook 235 provided on one of the first grill frame 121 and the inner frame 151, and a third hook-catching portion 236 provided on the other of the first grill frame 121 and the inner frame 151.

For example, the third hook 235 may be provided on the outer surface of the first grill frame 121, and the third hook-catching portion 236 may be provided on the outer surface of the inner frame 151.

The third hook 235 may be arranged adjacent to the third rotation-inducing groove 233 on the outer side of the inner frame 151. The third hook 235 may be spaced apart from the third rotation-inducing groove 233 in the inner frame 151 in the circumferential direction.

The third hook 235 may have a free end that is elastically deformable. The third hook 235 may be elastically deformed in a direction inclined with respect to the rotation assembly direction. The third hook 235 may include a third protrusion 2351 that protrudes toward the inner frame 151. The third protrusion 2351 may protrude downward.

The third hook-catching portion 236 may be provided such that the third hook 235 is inserted thereinto when the grill assembly 120 is rotated at a predetermined angle. When the third hook 235 is inserted into the third hook-catching portion 236, the third hook 235 may be restricted from moving in the rotation assembly direction by the third hook-catching portion 236.

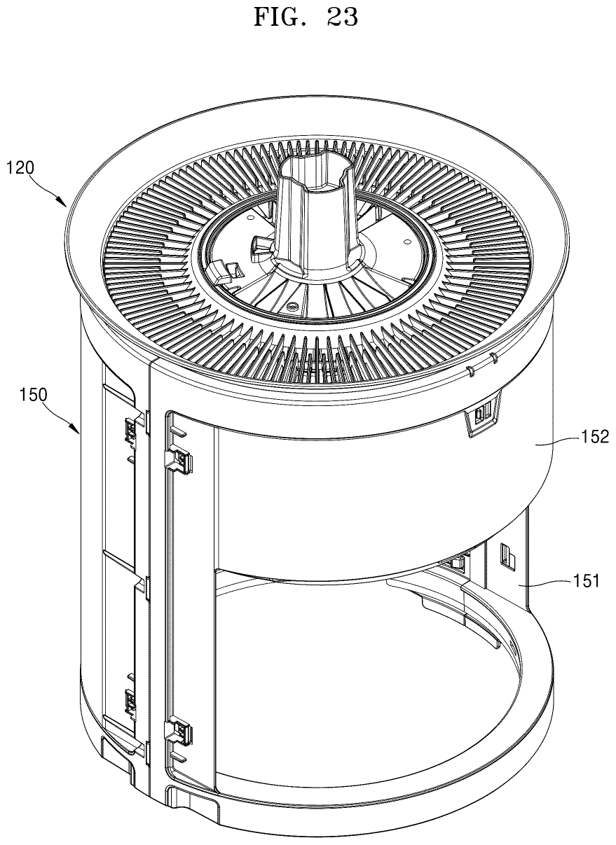

FIG. 23 is a drawing illustrating a state in which a grill assembly is assembled onto a body frame in the air purifier according to an embodiment of the disclosure.

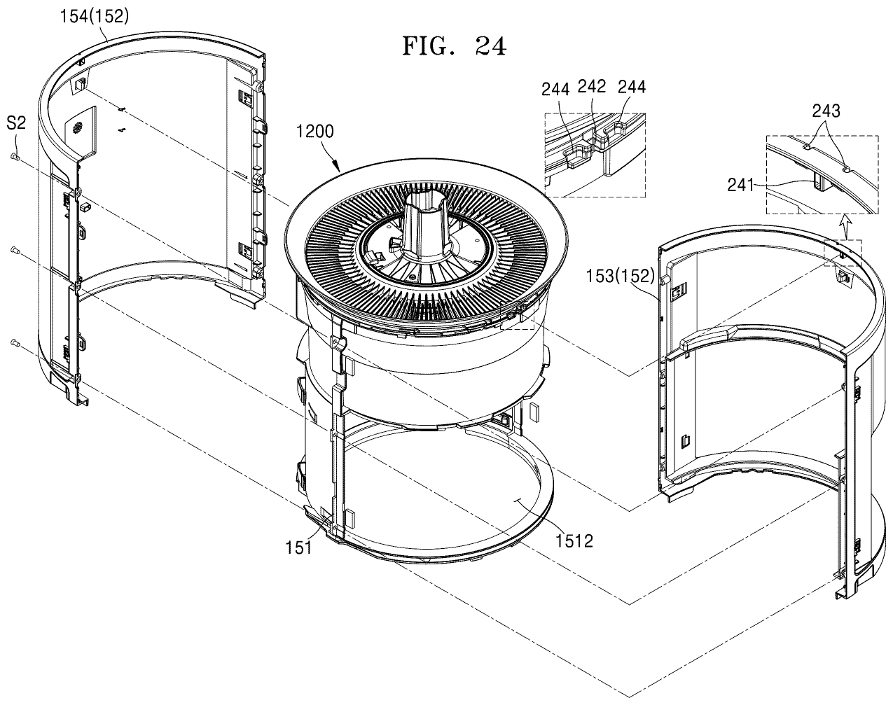

FIG. 24 is an exploded perspective view illustrating a state in which a cover frame is separated from an inner frame in the air purifier according to an embodiment of the disclosure.

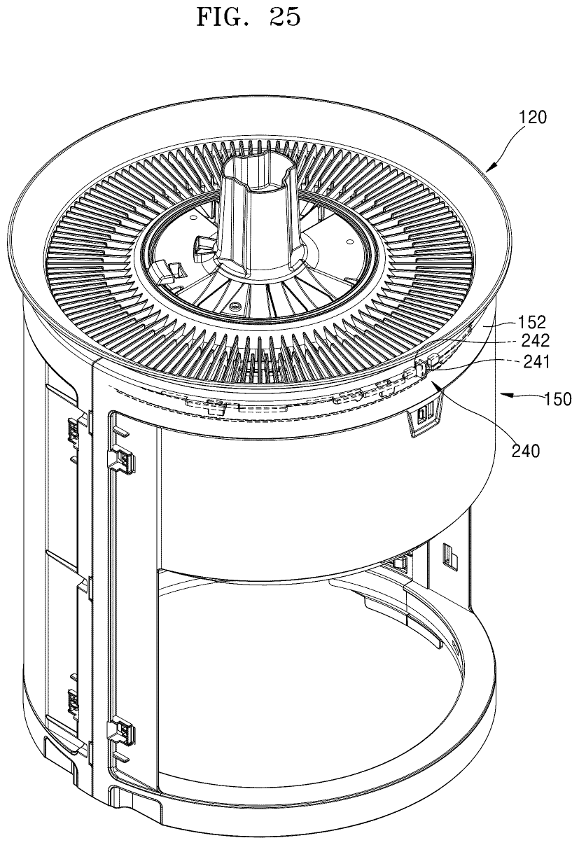

FIG. 25 is a drawing illustrating a state in which a cover frame is assembled onto an inner frame in an air purifier of FIG. 23 according to an embodiment of the disclosure.

Referring to FIGS. 23, 24, and 25, the cover frame 152 according to an embodiment may be configured to cover a side portion of the inner frame 151. A plurality of cover frames 152 may be provided. Each of the cover frames 152 may be configured to cover a portion of a side of the inner frame 151. For example, a front cover frame 152 may cover a side of the inner frame 151 in which an open surface 1512 is formed. A rear cover frame 152 may cover the other side of the inner frame 151. The front cover frame 152 and the rear cover frame 152 may be fixed by a fastening member S2.

At least one of the cover frame 152 and the grill assembly 120 may include a rotation prevention structure 240 that prevents rotation of the grill assembly 120 relative to the cover frame 152. For example, the rotation prevention structure 240 may include a rotation prevention protrusion 241 provided on one of the cover frame 152 and the grill assembly 120, and a rotation prevention groove 242 provided on the other of the cover frame 152 and the grill assembly 120. For example, the rotation prevention protrusion 241 may be provided on the inner surface of the cover frame 152, and the rotation prevention groove 242 into which the rotation prevention protrusion 241 is inserted may be provided on the outer surface of the grill assembly 120. When the cover frame 152 is coupled to the inner frame 151, the rotation prevention protrusion 241 may be inserted into the rotation prevention groove 242, and thus, the cover frame 152 may be restricted from rotating relative to the grill assembly 120.

The inner surface of the cover frame 152 may further include a structure for restricting rotation relative to the grill assembly 120. For example, an auxiliary rotation prevention protrusion 243 may be provided on the inner surface of the cover frame 152, and an auxiliary rotation prevention groove 244 into which the auxiliary rotation prevention protrusion 243 is inserted may be provided on the outer surface of the grill assembly 120. When the cover frame 152 is coupled to the inner frame 151, the auxiliary rotation prevention protrusion 243 of the cover frame 152 may be inserted into the auxiliary rotation prevention groove 244 of the grill assembly 120, and thus, the cover frame 152 may be restricted from rotating relative to the grill assembly 120.

The auxiliary rotation prevention protrusion 243 may be configured to be visible on the outer surface of the cover frame 152. For example, the auxiliary rotation prevention groove 244 of the cover frame 152 may have a protrusion shape that protrudes inwardly from the inner surface and a concave shape that is concave inwardly from the outer surface. In this way, the auxiliary rotation prevention protrusion 243 of the cover frame 152 that is visible may easily confirm the assembly position of the cover frame 152.

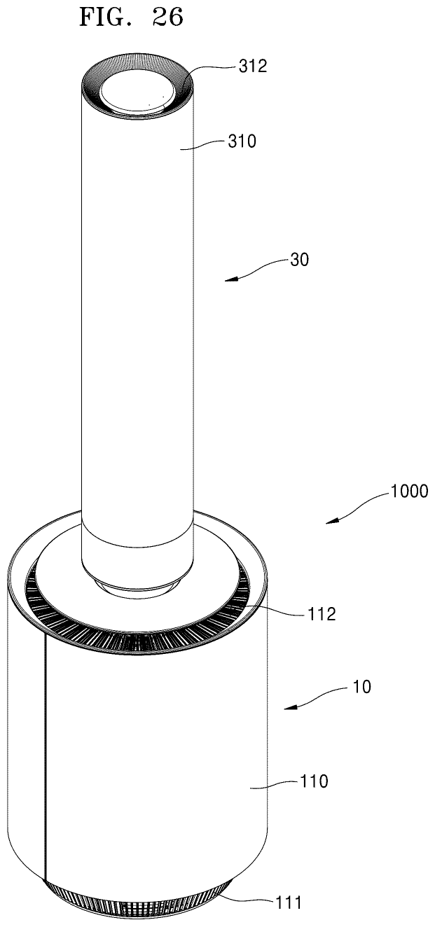

FIG. 26 is a perspective view illustrating an air purifying apparatus according to an embodiment of the disclosure.

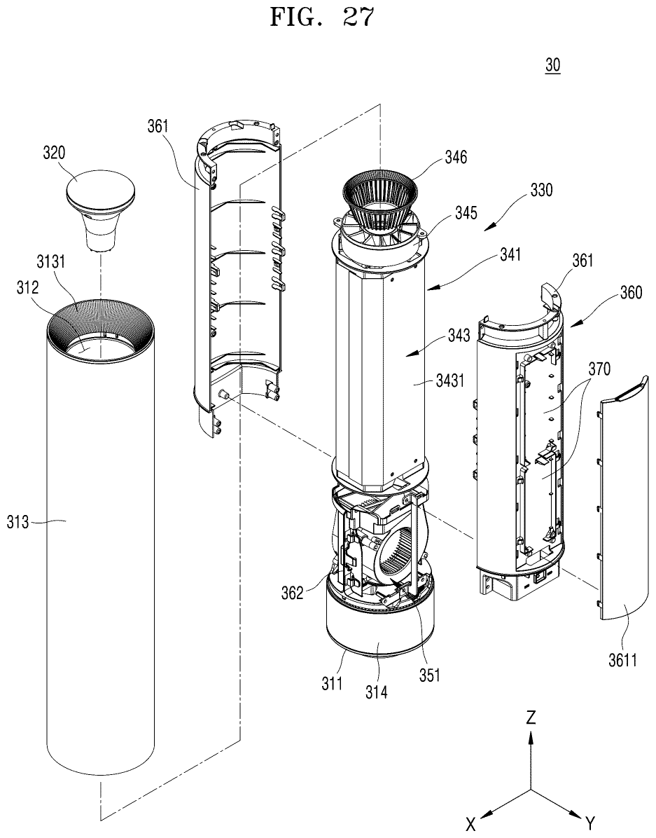

FIG. 27 is an exploded perspective view illustrating an air sterilizer of FIG. 26 according to an embodiment of the disclosure.

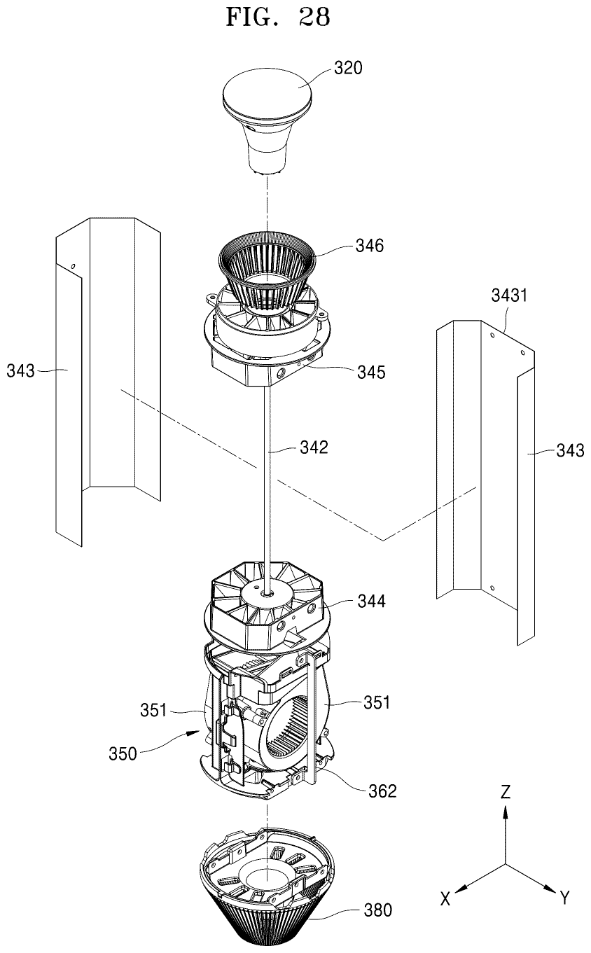

FIG. 28 is an exploded perspective view of a portion of an air sterilizer of FIG. 27 according to an embodiment of the disclosure.

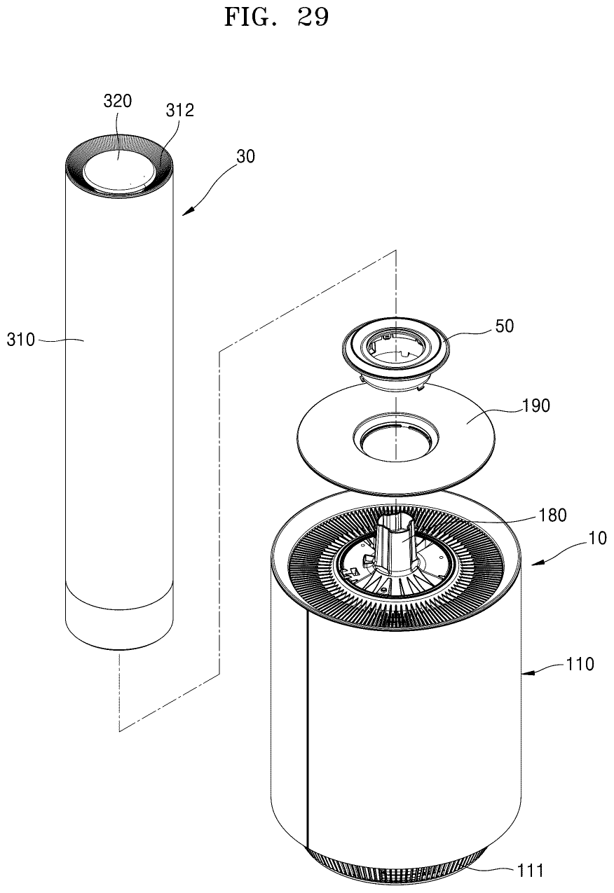

FIG. 29 is an exploded perspective view of a connection part of an air purifying apparatus of FIG. 26 according to an embodiment of the disclosure.

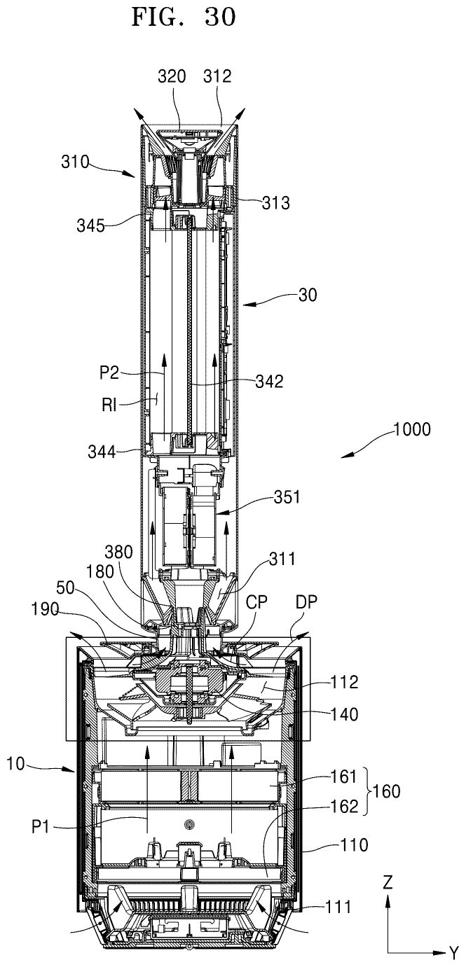

FIG. 30 is a cross-sectional view of an air purifying apparatus of FIG. 26 according to an embodiment of the disclosure.

Referring to FIGS. 26 to 30, the air purifying apparatus 1000 may be an apparatus designed to control air properties, such as temperature, humidity, cleanliness, or air flow. For example, the air purifying apparatus 1000 may be an apparatus for increasing the cleanliness of indoor air.

The air purifying apparatus 1000 according to an embodiment may include an air sterilizer 30 and the air purifier 10 according to the embodiment described above.

The air sterilizer 30 may be configured to sterilize air introduced from the outside, and discharge the sterilized air to the outside. For example, the air sterilizer 30 may include a second introduction port 311 and a second discharge port 312. The air sterilizer 30 may include a second flow path P2 provided between the second introduction port 311 and the second discharge port 312.

The air sterilizer 30 may include a housing 310 forming the exterior of the air sterilizer 30, and a main body module 330 arranged at least in part inside the housing 310. The main body module 330 may include an ultraviolet sterilization module 341 configured to generate ultraviolet rays, and a second fan 350 configured to form an air flow toward the ultraviolet sterilization module 341.

The ultraviolet sterilization module 341 may be arranged on the second flow path P2. The ultraviolet sterilization module 341 may include at least one ultraviolet light source 342 provided on the second flow path P2. The ultraviolet light source 342 generates ultraviolet rays, and a plurality of ultraviolet light sources 342 may be provided.

The ultraviolet light source 342 may be arranged inside the housing 310, and may sterilize air moving along the second flow path P2. The ultraviolet light source 342 may emit ultraviolet rays toward air introduced through the second introduction port 311. The ultraviolet light source 342 may generate ultraviolet-C (UVC) to remove bacteria contained in the air. The ultraviolet light source 342 may generate light with a wavelength of 220 nm to 280 nm. The ultraviolet light source 342 may remove contaminants, such as bacteria or viruses in the introduced air by emitting ultraviolet rays toward the introduced air.

The ultraviolet sterilization module 341 may include a reflective cover 343 designed to reflect ultraviolet rays emitted from the ultraviolet light source 342. The reflective cover 343 may cover an outer side of the ultraviolet light source 342. The reflective cover 343 may be provided in the second flow path P2.

At least a portion of the reflective cover 343 may include a metal material such that the inner surface of the reflective cover 343 facing the ultraviolet light source 342 reflects the ultraviolet rays. For example, the inner surface of the reflective cover 343 may include a material with high light reflectivity. For example, the inner surface of the reflective cover 343 may be plated with a metal, such as chrome. As another example, the entirety of the reflective cover 343, including its inner surface, may be configured as a plate made of a metal material with high light reflectivity.

The reflective cover 343 may have a curved shape. For example, the reflective cover 343 may include a plurality of flat plates, and the plurality of plate shapes may be arranged with certain angles therebetween to surround the ultraviolet light source 342.

The reflective cover 343 may have a polygonal cross-sectional structure. For example, the cross-sectional shape of the reflective cover 343 may be designed considering the arrangement of a second printed circuit board assembly 370 to be described below. For example, the cross-sectional shape of the reflective cover 343 may be an asymmetrical shape in which a plate 3431 facing the second printed circuit board assembly 370 is relatively larger than the other plates.

A plurality of reflective covers 343 may be provided. For example, the plurality of reflective covers 343 may each cover an outer side of the ultraviolet light source 342. For example, some of the plurality of reflective covers 343 may cover one side of the ultraviolet light source 342, and the other reflective covers 343 may cover the other side of the ultraviolet light source 342. However, the shape and number of reflective covers 343 are not limited thereto, and may be variously modified such that ultraviolet rays emitted from the ultraviolet light source 342 are reflected. For example, only one reflective cover 343 may be provided. For example, the reflective cover 343 may be manufactured by bending a single plate.

The air sterilizer 30 may include ultraviolet ray blocking members 344 and 345 provided to prevent ultraviolet rays emitted from the ultraviolet light source 342 from being incident on areas outside an irradiation area RI.

The ultraviolet ray blocking members 344 and 345 may be provided in the second flow path P2. The ultraviolet ray blocking member 344 and 345 may be formed to allow air flowing along the second flow path P2 to pass therethrough. The ultraviolet ray blocking members 344 and 345 may be provided to prevent the ultraviolet rays emitted from the ultraviolet light source 342 from being emitted to the outside of the irradiation area RI while allowing air to flow along the second flow path P2.

Through the ultraviolet ray blocking members 344 and 345, the internal parts of the air purifier provided outside the ultraviolet sterilization module 341 may be prevented from being damaged by ultraviolet rays, and the ultraviolet rays may be prevented from being emitted to the outside of the air purifier through the second discharge port 312.

The second fan 350 is for generating a flow inside the air sterilizer 30 and may be provided in the second flow path P2. The second fan 350 may be configured to form an air flow such that air introduced into the air sterilizer is directed toward the ultraviolet sterilization module 341.

The ultraviolet sterilization module 341 and the second fan 350 may be arranged along the second flow path P2. For example, the ultraviolet sterilization module 341 and the second fan 350 may be arranged in a first direction Z. For example, the second fan 350 may be arranged below the ultraviolet sterilization module 341. The second fan 350 may be arranged between the second introduction port 311 and the ultraviolet sterilization module 341. When the second fan 350 is driven, air introduced through the second introduction port 311 may sequentially pass through the second fan 350 and the ultraviolet sterilization module 341 to be discharged through the second discharge port 312. However, the arrangement of the second fan 350 is not limited thereto, and the second fan 350 may be arranged above the ultraviolet sterilization module 341.

The second fan 350 may include a plurality of blowers 351. The plurality of blowers 351 may be centrifugal blowers. The plurality of blowers 351 may have shapes corresponding to each other. The plurality of blowers 351 may be arranged symmetrically around the central axis of the housing 310.

Each of the plurality of blowers 351 may be arranged to discharge air toward the ultraviolet sterilization module 341. The direction in which air is discharged from some of the plurality of blowers 351, which is inclined with respect to the first direction Z may be arranged to be opposite to the direction in which air is discharged from the other blowers 351, which is inclined with respect to the first direction Z.

However, the shape, arrangement, number, and the like of the blowers 351 of the second fan 350 are not limited thereto and may vary. For example, the blower 351 may be another type of blower 351, such as an axial-flow-type blower, or only one blower 351 may be provided.

The main body module 330 may further include an inner frame 360. The inner frame 360 may be arranged inside the housing 310. The inner frame 360 may be provided to support at least some of the parts of the air sterilizer 30.

For example, the inner frame 360 may include a first support frame 361 that supports the ultraviolet sterilization module 341. The first support frame 361 may support the second printed circuit board assembly 370 and a control panel 320. The first support frame 361 may include a board mounting cover 3611 that covers the second printed circuit board assembly 370.

The inner frame 360 may further include a second support frame 362 that supports the second fan 350. The second support frame 362 may be coupled to the first support frame 361. However, the configuration of the inner frame 360 is not limited thereto, and the first support frame 361 and the second support frame 362 may be configured as one body, or the first support frame 361 or the second support frame 362 may be omitted.

The housing 310 forms at least a portion of the exterior of the air sterilizer 30, and the ultraviolet sterilization module 341 and the second fan 350 may be arranged inside the housing 310. The housing 310 may include the second introduction port 311 through which air is introduced, and the second discharge port 312 through which air is discharged.

The housing 310 may have a hollow structure that provides a space for accommodating at least a portion of the main body module 330. The housing 310 may have a cylindrical structure. The ultraviolet sterilization module 341 and the second fan 350 may be arranged inside the housing 310.

The housing 310 may have a length extending in the first direction Z, for example, in the vertical direction. For example, the housing 310 may have a shape in which the length in the first direction Z is greater than the width in a second direction Y that is perpendicular to the first direction Z. For example, the length of the housing 310 in the first direction Z may be three to seven times the width of the housing 310 in the second direction Y.

The housing 310 may be separated into an upper case 313 and a lower case 314. The second discharge port 312 may be provided in the upper case 313, and the second introduction port 311 may be provided in the lower case 314. For example, the second discharge port 312 may be provided at an end of the upper case 313, for example, at the upper end, and the second introduction port 311 may be provided at an end of the lower case 314, for example, at the lower end. An upper cover 3131 in which the second discharge port 312 is provided may be arranged in an upper portion of the upper case 313.

The control panel 320 may be arranged at an end of the upper case 313 to overlap the second discharge port 312 in the first direction Z. The control panel 320 may be arranged at a certain distance from the second discharge port 312 such that air is discharged through the second discharge port 312. The control panel 320 may be supported on the inner frame 360 by an outlet grill 346.

A second coupling unit 380 for supporting the second fan 350 may be arranged inside the lower case 314. The second fan 350 and the second support frame 362 may be arranged on the second coupling unit 380. The second coupling unit 380 may be configured to be coupled to the air purifier.

The air purifier 10 may be assembled to be connected to the air sterilizer 30. The air purifier 10 may be detachably assembled to the air sterilizer 30. For example, the air purifier 10 may include a first unit coupling portion 180 provided to be coupled to the air sterilizer 30. For example, the first unit coupling portion 180 may be coupled to a portion of the second coupling unit 380 provided in a lower portion of the air sterilizer 30, such that the air purifier and the air sterilizer 30 may be connected to each other.

The air purifying apparatus 1000 may include an intermediate duct 50. The intermediate duct 50 may be provided between the housing 110 of the air purifier 10 and the housing 310 of the air sterilizer 30. The intermediate duct 50 may connect the housing 110 of the air purifier 10 to the housing 310 of the air sterilizer 30.

The intermediate duct 50 may be in communication with each of the discharge port 112 and the second introduction port 311. The intermediate duct 50 may be provided such that at least a portion of air discharged from the discharge port 112 flows into the second introduction port 311. The air sterilizer 30 may have the second introduction port 311 connected to the air purifier 10, and accordingly, air from which dust has been removed by the air purifier 10 may be sterilized by the ultraviolet sterilization module 341 of the air sterilizer 30.

A connection flow path CP may be provided between the second discharge port 312 of the air sterilizer 30 and the second introduction port 311 of the air purifier 10. A fixed guard 190 may guide at least a portion of air discharged through the discharge port 112, excluding the air that flows into the second flow path P2 through the connection flow path CP, to flow along a discharge flow path DP when the fan 130 of the air purifier 10 and the second fan 350 of the air sterilizer 30 are driven simultaneously. For example, the fixed guard 190 may cover at least a portion of the discharge port 112 from the outside.

In the air purifying apparatus 1000 according to the above-described embodiment of the disclosure, at least a portion of the air from which foreign substances have been removed by the air purifier 10 may be transferred to the air sterilizer 30. The air transferred to the air sterilizer 30 is sterilized by the ultraviolet light source 342 of the ultraviolet sterilization module 341 and then discharged to the outside through the second discharge port 312.

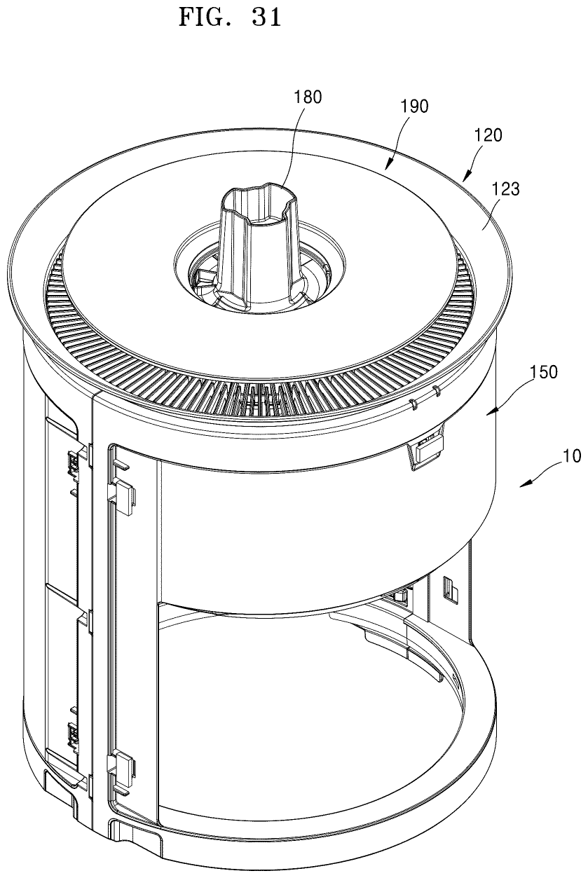

FIG. 31 is a perspective view illustrating an air purifier having a fixed guard according to an embodiment of the disclosure.

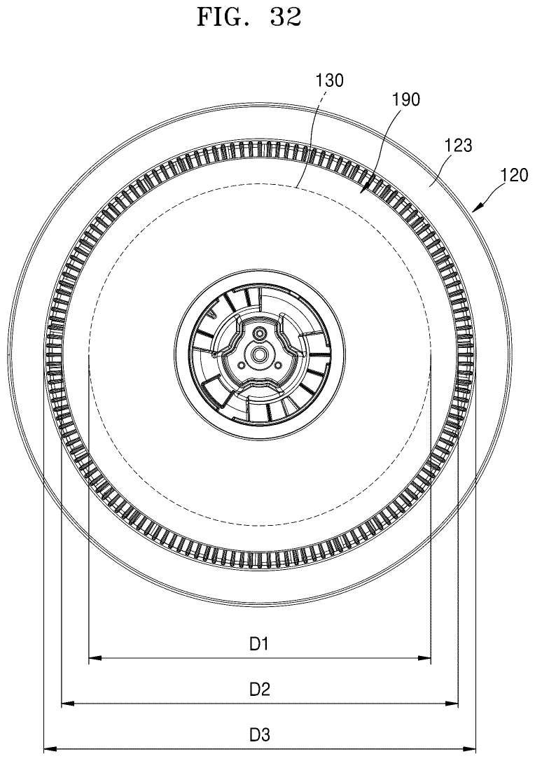

FIG. 32 is a plan view of an air purifier of FIG. 31 as viewed from a top of an air purifier according to an embodiment of the disclosure.

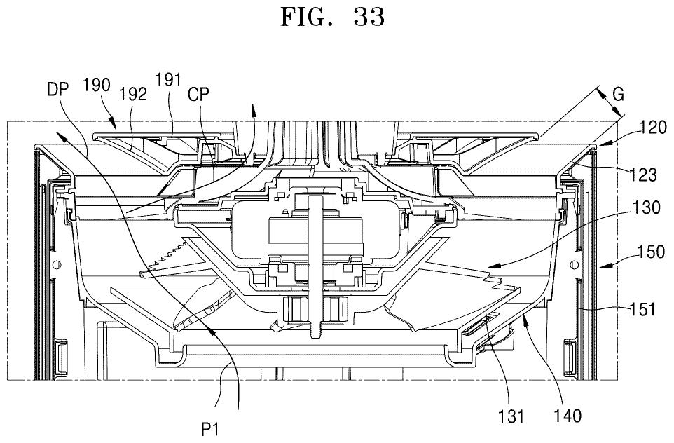

FIG. 33 is a cross-sectional view of a portion of an air purifier of FIG. 31 according to an embodiment of the disclosure.

Referring to FIGS. 31, 32, and 33, the air purifier 10 according to an embodiment may further include a fixed guard 190 for protecting a grill assembly 120. The fixed guard 190 may be arranged above the grill assembly 120 to be spaced apart from the grill assembly 120 by a predetermined distance.

The fixed guard 190 may be arranged to overlap a portion of the grill assembly 120 in the vertical direction. A diameter D2 of the fixed guard 190 may be greater than a diameter D1 of a fan 130 The diameter D2 of the fixed guard 190 may be less than the diameter of the grill assembly 120. The diameter D2 of the fixed guard 190 may be less than an inner diameter D3 of an edge frame 123. Accordingly, the fixed guard 190 may reduce the fan 130 located below the grill assembly 120 from being visible to the outside. In addition, the fixed guard 190 may protect the grill assembly 120 while allowing airflow through the grill assembly 120 by being partially disposed on the grill assembly 120.

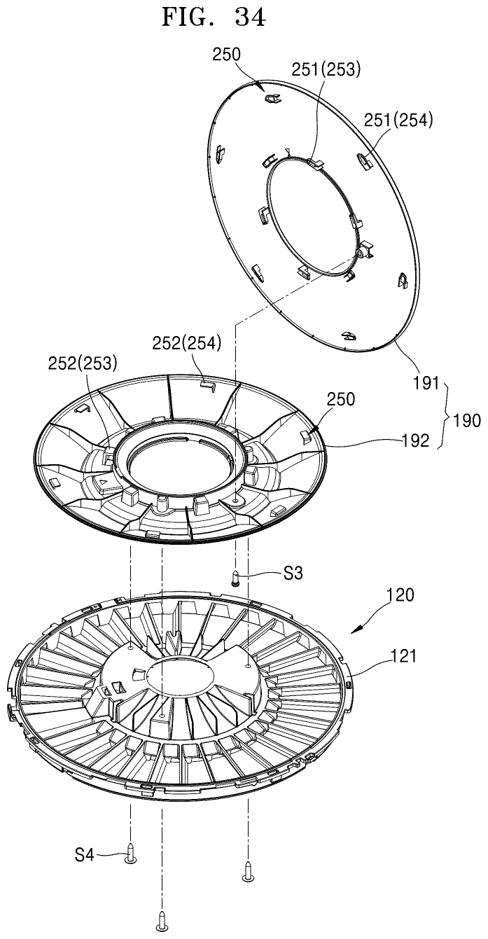

FIG. 34 is an exploded perspective view of a fixed guard according to an embodiment of the disclosure.

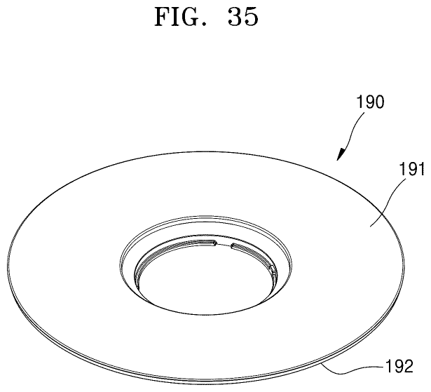

FIG. 35 is an assembled perspective view of a fixed guard according to an embodiment of the disclosure.

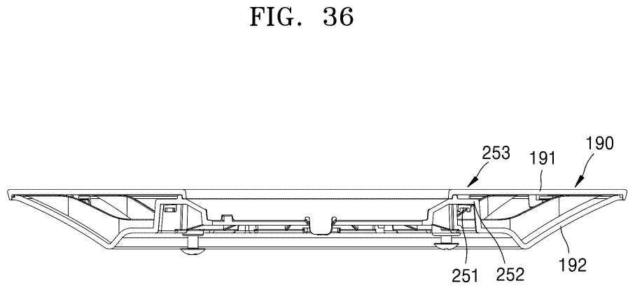

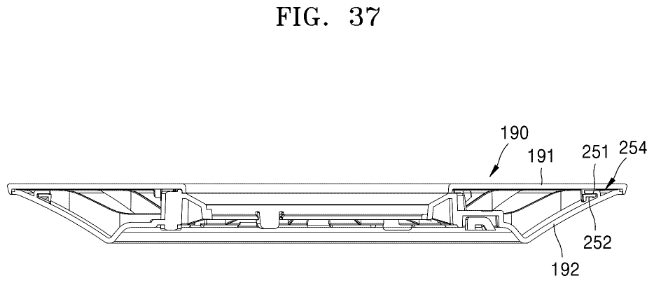

FIGS. 36 and 37 illustrate cross-sectional views of a fixed guard according to various embodiments of the disclosure.

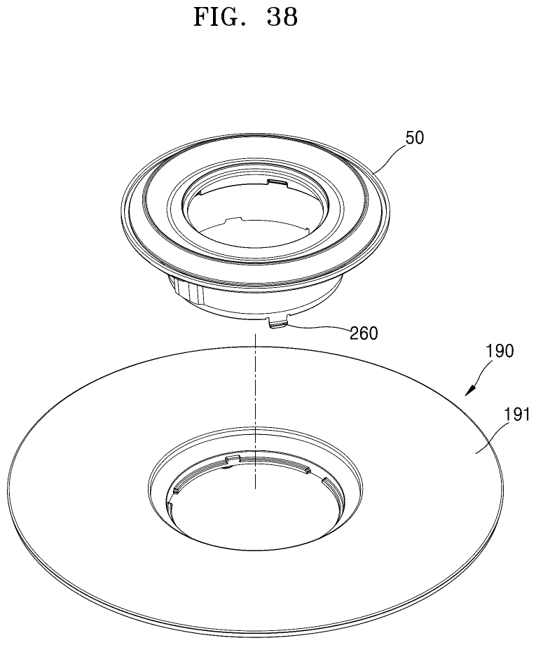

FIG. 38 is an exploded perspective view illustrating a state in which a fixed guard is separated from an intermediate duct according to an embodiment of the disclosure.

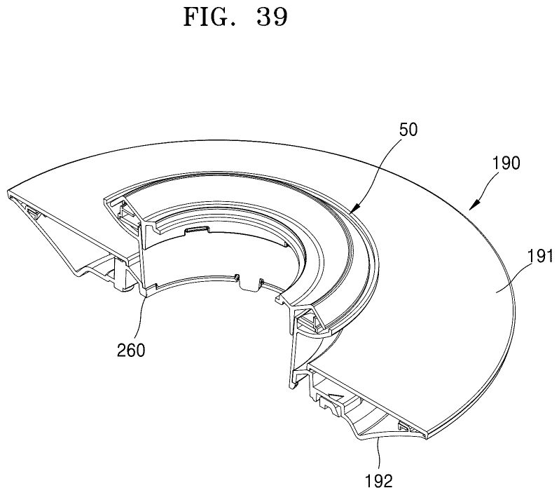

FIG. 39 is a cross-sectional perspective view illustrating a state in which a fixed guard is assembled to an intermediate duct according to an embodiment of the disclosure.

Referring to FIGS. 33, 34, and 35, the fixed guard 190 according to an embodiment may include an upper guard 191 and a lower guard 192. The upper guard 191 may form at least a portion of an upper exterior of the air purifier 10. The diameter of the upper guard 191 may be greater than the diameter of a fan 130.

The lower guard 192 may guide a flow path of air discharged from a grill assembly 120. For example, the lower guard 192 may form a discharge flow path DP through which at least a portion of air discharged from a discharge port 112 is discharged to the outside of the air purifier 10. The discharge flow path DP may branch from a first flow path P1 and extend in an outer direction of the air purifier 10. For example, the outer surface of the lower guard 192 may guide air moving along the discharge flow path DP.

The lower guard 192 may form at least a portion of a connection flow path CP. The connection flow path CP may extend in a different direction from the discharge path DP. For example, the discharge path DP may extend upwardly in an outward direction, and the connection flow path CP may extend upwardly in an inward direction. For example, the inner surface of the lower guard 192 may guide air moving along the connection flow path CP.

The fixed guard 190 may be configured to function as a handle of the air purifier 10. For example, the fixed guard 190 may be arranged such that a user's finger may enter between the fixed guard 190 and the grill assembly 120. For example, a gap G between the fixed guard 190 and the grill assembly 120 may be 21 mm or more. The gap between the lower guard 192 and the edge frame 123 may be 21 mm or more. The gap G between the fixed guard 190 and the grill assembly 120 may be 50 mm or less.

Referring to FIGS. 34 to 37, the upper guard 191 may be detachably assembled to the lower guard 192. For example, the upper guard 191 and the lower guard 192 may be configured to be restricted from moving in the vertical direction by a hook structure 250. For example, a hook 251 may be provided on one of the upper guard 191 and the lower guard 192, and a hook-catching portion 252 to which the hook 251 is coupled may be provided on the other of the upper guard 191 and the lower guard 192. For example, the hook 251 may be provided on the lower surface of the upper guard 191, and the hook-catching portion 252 to which the hook 251 is coupled may be provided on the upper surface of the lower guard 192.

After the upper guard 191 is arranged to face the lower guard 192 such that the hook 251 faces the hook-catching portion 252, the upper guard 191 may be rotated relative to the lower guard 192. During the process of rotating the upper guard 191 relative to the lower guard 192, the hook 251 may be inserted into the hook-catching portion 252. The hook 251 inserted into the hook-catching portion 252 may be restricted from moving in the vertical direction.

A plurality of hook structures 250 may be provided in a radial direction of the fixed guard 190. For example, the hook structure 250 may include an inner hook structure 253 adjacent to the inner edge of the fixed guard 190 and an outer hook structure 254 adjacent to the outer edge of the fixed guard 190.

Referring to FIG. 36, the inner hook structure 253 may include a hook 251 adjacent to the inner edge of the upper guard 191 and a hook-catching portion 252 adjacent to the inner edge of the lower guard 192. As the hook 251 and the hook-catching portion 252 of the inner hook structure 253 are interlocked with each other, the upper guard 191 and the lower guard 192 may be prevented from being separated from each other in the vertical direction at an inner edge portion.

Referring to FIG. 37, the outer hook structure 254 may include a hook 251 adjacent to the outer edge of the upper guard 191 and a hook-catching portion 252 adjacent to the outer edge of the lower guard 192. As the hook 251 and the hook-catching portion 252 of the outer hook structure 254 are interlocked with each other, the upper guard 191 and the lower guard 192 may be prevented from being separated from each other in the vertical direction at an outer edge portion.

As described above, by the plurality of hooks 251 and hook-catching portions 252 provided in the radial direction, when an external force is applied to the fixed guard 190, the gap between the upper guard 191 and the lower guard 192 may be prevented from widening.

In the above-described embodiment of the disclosure, the hook 251 is provided on the upper guard 191 and the hook-catching portion 252 is provided on the lower guard 192. However, the arrangement of the hook 251 and the hook-catching portion 252 is not limited thereto and may vary.

Referring back to FIG. 34, the upper guard 191 and the lower guard 192 assembled by the hook structure 250 may be fixed by a fastening member S3. For example, when the fastening member S3 is fastened from the bottom to the top, the upper guard 191 and the lower guard 192 may be fixed. Accordingly, the upper guard 191 may be prevented from rotating relative to the lower guard 192.

The fixed guard 190 may be fixed to a first grill frame 121 of the grill assembly 120. For example, the fixed guard 190 may be fixed to the first grill frame 121 by a fastening member S4 fastened from bottom to top.

Referring to FIGS. 38 and 39, an intermediate duct 50 may be assembled to the fixed guard 190 of the air purifier 10 according to an embodiment of the disclosure. The intermediate duct 50 may be assembled to the inner surface of the fixed guard 190. For example, a hook 260 arranged in a lower portion of the intermediate duct 50 may be assembled to the inner surface of the lower guard 192. For example, in the process of inserting the intermediate duct 50 from the top to the bottom toward the inner surface of the lower guard 192, the hook 260 may be assembled to the lower guard 192.

For the purposes of promoting understanding of the disclosure, reference numerals are used in the preferred embodiments illustrated in the drawings, and particular terms are used to describe the embodiments of the disclosure, however, the disclosure is not limited by the terms and should be construed to encompass all components that would normally occur to those skilled in the art.

Particular executions described herein are merely examples and do not limit the scope of the disclosure in any way. For the sake of brevity, related-art electronics, control systems, software and other functional aspects of the systems may not be described in detail. Furthermore, line connections or connection members between elements depicted in the drawings represent functional connections and/or physical or circuit connections by way of example, and in actual applications, they may be replaced or embodied with various suitable additional functional connections, physical connections, or circuit connections. In addition, no item or component is essential to the practice of the disclosure unless the item or component is specifically described as being “essential” or “critical”. As used herein, the term, such as “comprising”, “including” and the like are used to be understood as being an open-ended term for description.

The term ‘the’ and other demonstratives similar thereto in the specification of the disclosure (especially in the following claims) should be understood to include a singular form and plural forms. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the operations of the methods described herein may be performed in any suitable order unless otherwise indicated herein or otherwise clearly contradicted by context. The disclosure is not limited to the described order of the operations. The use of any and all examples, or language (e.g., ‘and the like’) provided herein, is intended merely to better illuminate the disclosure and does not pose a limitation on the scope of the disclosure unless otherwise claimed. Furthermore, various changes and modifications will be readily apparent to one of ordinary skill in the art without departing from the spirit and scope of the disclosure.

An air purifier and air purifying apparatus according to embodiments may provide an assembly structure that minimizes the use of fastening members, such as screws. Through this, the assembly process may be simplified, and the assembly time and material and labor costs for assembly may be reduced.

An air purifier according to an embodiment may include a fan that generates air flow, a grill assembly that is arranged above the fan and forms an upper exterior of the air purifier, and a body frame configured to support the grill assembly, wherein the grill assembly may include a first grill frame having first grills arranged at first intervals, a second grill frame having second grills arranged at second intervals that are smaller than the first intervals, and an edge frame that overlaps an outer edge of the second grill frame in a vertical direction and is assembled to an outer surface of the first grill frame, and wherein the grill assembly may have a first rotation assembly structure configured to rotationally assemble the edge frame to the first grill frame such that the edge frame is fixed in the vertical direction with respect to the first grill frame and rotational movement is restricted with respect to the first grill frame.

The first rotation assembly structure may include a first separation prevention structure for preventing separation in the vertical direction of the edge frame and a first rotation prevention structure for preventing rotation of the edge frame.

The first separation prevention structure may include a first rotation-inducing protrusion provided on one of the first grill frame and the edge frame, and a first rotation-inducing groove provided on the other of the first grill frame and the edge frame and allowing rotational movement of the first rotation-inducing protrusion, but restricting movement in the vertical direction perpendicular to the rotational movement.

The first rotation prevention structure may include a first hook provided on one of the first grill frame and the edge frame and capable of elastic deformation, and a first hook-catching portion provided on the other of the first grill frame and the edge frame and provided such that the first hook is inserted into the first hook-catching portion when the edge frame is positioned at a predetermined angle.

The air purifier may further include a duct frame assembled to an outer surface of the grill assembly and arranged to accommodate the fan, wherein at least one of the grill assembly and the duct frame may have a second rotation assembly structure configured to rotationally assemble the grill assembly to the duct frame such that the grill assembly is fixed in the vertical direction with respect to the duct frame and rotational movement is restricted with respect to the duct frame.

A rotation assembly direction of the first rotation assembly structure and a rotation assembly direction of the second rotation assembly structure may be the same.

At least one of the grill assembly and the body frame may have a third rotation assembly structure configured to rotationally assemble the grill assembly to the body frame such that the grill assembly is fixed in the vertical direction with respect to the body frame and rotational movement is restricted with respect to the body frame.

A rotation assembly direction of the first rotation assembly structure and a rotation assembly direction of the second rotation assembly structure may be the same.

The body frame may include an inner frame that accommodates the duct frame and supports the grill assembly, and a cover frame that covers a side of the body frame.

The cover frame may include a front cover frame that covers one side of the inner frame, and a rear cover frame that covers the other side of the inner frame, wherein the front cover frame and the rear cover frame may be fixed to surround the side of the inner frame by a fastening member, and at least one of the cover frame and the grill assembly may include a rotation prevention structure that prevents rotation of the grill assembly relative to the cover frame.

The air purifier may further include a fixed guard overlapping the grill assembly in the vertical direction to protect the grill assembly and arranged above the grill assembly to be spaced apart from the grill assembly by a predetermined distance.

A diameter of the fixed guard may be greater than a diameter of the fan and less than a diameter of the grill assembly.

A gap between the fixed guard and the grill assembly may be 21 mm or more.

The fixed guard may include an upper guard forming a portion of an upper exterior of the air purifier, and a lower guard guiding a flow path of air discharged from the grill assembly, wherein the upper guard and the lower guard may be assembled by a hook structure, and the hook structure may include an inner hook structure adjacent to an inner edge of the fixed guard, and an outer hook structure adjacent to an outer edge of the fixed guard.

An air purifying apparatus according to an embodiment may include an air purifier configured to remove dust in air, and an air sterilizer configured to sterilize the air from which the dust has been removed.

The air purifier and air purification device according to embodiments may simplify the assembly process by minimizing the use of fastening members, such as screws, and may reduce the assembly time and material and labor costs for assembly.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage, such as, for example, a storage device like read only memory (ROM), whether erasable or rewritable or not, or in the form of memory, such as, for example, random access memory (RAM), memory chips, device or integrated circuits or on an optically or magnetically readable medium, such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An air purifier comprising:
a fan that generates airflow;
a grill assembly arranged above the fan and forms an upper exterior of the air purifier; and
a body frame configured to support the grill assembly,
wherein the grill assembly comprises:
a first grill frame having first grills arranged at first intervals (G1),
a second grill frame having second grills arranged at second intervals (G2) that are smaller than the first intervals, and
an edge frame overlapping an outer edge of the second grill frame in a vertical direction and being assembled onto an outer surface of the first grill frame, and
wherein the grill assembly has a first rotation assembly structure configured to rotationally assemble the edge frame onto the first grill frame such that the edge frame is fixed in the vertical direction with respect to the first grill frame and rotational movement is restricted with respect to the first grill frame.

2. The air purifier of claim 1, wherein the first rotation assembly structure comprises a first separation prevention structure for preventing separation of the edge frame in the vertical direction and a first rotation prevention structure for preventing rotation of the edge frame.

3. The air purifier of claim 2, wherein the first separation prevention structure comprises:
a first rotation-inducing protrusion provided on one of the first grill frame and the edge frame; and
a first rotation-inducing groove provided on the other of the first grill frame and the edge frame and allowing rotational movement of the first rotation-inducing protrusion, but restricting movement in the vertical direction perpendicular to the rotational movement.

4. The air purifier of claim 2, wherein the first rotation prevention structure comprises:
a first hook provided on one of the first grill frame and the edge frame and capable of elastic deformation; and
a first hook-catching portion provided on the other of the first grill frame and the edge frame and provided such that the first hook is inserted into the first hook-catching portion when the edge frame is positioned at a predetermined angle.

5. The air purifier of claim 1, further comprising:
a duct frame assembled onto an outer surface of the grill assembly and arranged to accommodate the fan,
wherein at least one of the grill assembly and the duct frame has a second rotation assembly structure configured to rotationally assemble the grill assembly onto the duct frame such that the grill assembly is fixed in the vertical direction with respect to the duct frame and rotational movement is restricted with respect to the duct frame.

6. The air purifier of claim 5, wherein a rotation assembly direction of the first rotation assembly structure and a rotation assembly direction of the second rotation assembly structure are the same.

7. The air purifier of claim 6, wherein at least one of the grill assembly and the body frame has a third rotation assembly structure configured to rotationally assemble the grill assembly onto the body frame such that the grill assembly is fixed in the vertical direction with respect to the body frame and rotational movement is restricted with respect to the body frame.

8. The air purifier of claim 7, wherein a rotation assembly direction of the first rotation assembly structure and a rotation assembly direction of the second rotation assembly structure are the same.

9. The air purifier of claim 8, wherein the body frame comprises an inner frame accommodating the duct frame and supporting the grill assembly, and a cover frame covering a side of the body frame.

10. The air purifier of claim 9,
wherein the cover frame comprises a front cover frame that covers one side of the inner frame, and a rear cover frame that covers the other side of the inner frame,
wherein, by means of a fastening member, the front cover frame and the rear cover frame are fixed to surround the side of the inner frame, and
wherein at least one of the cover frame and the grill assembly comprises a rotation prevention structure that prevents rotation of the grill assembly relative to the cover frame.

11. The air purifier of claim 1, further comprising:
a fixed guard overlapping the grill assembly in the vertical direction to protect the grill assembly, and arranged above the grill assembly to be spaced apart from the grill assembly by a predetermined distance.

12. The air purifier of claim 11, wherein a diameter (D2) of the fixed guard is greater than a diameter (D1) of the fan and less than a diameter (D3) of the grill assembly.

13. The air purifier of claim 11, wherein a gap (G) between the fixed guard and the grill assembly is 21 mm or more.

14. The air purifier of claim 13,
wherein the fixed guard comprises an upper guard forming a portion of an upper exterior of the air purifier, and a lower guard guiding a flow of air discharged from the grill assembly, and
wherein the upper guard and the lower guard are assembled by a hook structure, and the hook structure comprises an inner hook structure adjacent to an inner edge of the fixed guard, and an outer hook structure adjacent to an outer edge of the fixed guard.

15. An air purifying apparatus comprising an air purifier configured to remove dust from air and an air sterilizer configured to sterilize the air from which the dust has been removed, the air purifier comprising:
a fan generating airflow;
a grill assembly arranged above the fan and forming an upper exterior of the air purifier; and
a body frame configured to support the grill assembly,
wherein the grill assembly comprises:
a first grill frame having first grills arranged at first intervals,
a second grill frame having second grills arranged at second intervals that are smaller than the first intervals, and
an edge frame overlapping an outer edge of the second grill frame in a vertical direction and being assembled onto an outer edge of the first grill frame, and
wherein the grill assembly has a first rotation assembly structure configured to rotationally assemble the edge frame onto the first grill frame such that the edge frame is fixed in the vertical direction with respect to the first grill frame and rotational movement is restricted with respect to the first grill frame.
